(19) United States
(12) Patent Application Publication
(10) Pub. No.: US 2024/0228407 A1
(43) Pub. Date: Jul. 11, 2024

(54) INTEGRATION FOR FEED DILUTION IN OXIDATIVE DEHYDROGENATION (ODH) REACTOR SYSTEM

(71) Applicant: NOVA CHEMICALS (INTERNATIONAL) S.A., Fribourg (CH)

(72) Inventors: Mohammad KESHTKAR, Calgary (CA); David GENT, Red Deer (CA)

(21) Appl. No.: 18/288,767

(22) PCT Filed: Apr. 26, 2022
(86) PCT No.: PCT/IB2022/053873

§ 371 Date: Oct. 27, 2023

Related U.S. Application Data
(60) Provisional application No. 63/181,086, filed on Apr. 28, 2021.

(51) Int. Cl. C07C 11/04; C07C 5/48; C07C 51/215; C07C 53/08

(52) U.S. Cl. CPC C07C 11/04; C07C 5/48; C07C 51/215; C07C 53/08

(57) ABSTRACT

A system and method for producing ethylene, including dehydrogenating ethane to ethylene via an ODH catalyst in the presence of oxygen in an ODH reactor, discharging an effluent (including at least ethylene, water, and acetic acid) from the ODH reactor, recovering heat from the effluent for processing feed including ethane for the ODH reactor, recovering water from the effluent as recycle water for addition to the feed in performing water dilution of the feed, and adding oxygen to the feed to give a mixed feed including ethane, oxygen, and recycle water to the ODH reactor.

### CLAIM OF PRIORITY

This application claims priority to U.S. Provisional Application No. 63/181,086 filed on Apr. 28, 2021, the entire contents of which are hereby incorporated by reference.

### TECHNICAL FIELD

This disclosure relates to oxidative dehydrogenation (ODH) to produce ethylene.

### BACKGROUND ART

Catalytic oxidative dehydrogenation of alkanes into corresponding alkenes is an alternative to steam cracking. In contrast to steam cracking, oxidative dehydrogenation (ODH) may operate at lower temperature and generally does not produce coke. For ethylene production, ODH can provide a greater yield for ethylene than does steam cracking. The ODH may be performed in a reactor vessel having catalyst for the conversion of an alkane to a corresponding alkene. Acetic acid may be generated in the conversion of the lower alkanes (e.g., ethane) into the corresponding alkenes (e.g., ethylene).

Carbon dioxide is the primary greenhouse gas emitted through human activities. Carbon dioxide (CO₂) may be generated in various industrial and chemical plant facilities, including ODH facilities. At such facilities, more efficient utilization of energy may reduce CO₂ emissions at the facility and therefore decrease the CO₂ footprint of the facility.

### SUMMARY OF INVENTION

An aspect relates to a method of producing ethylene including adding water to ethane to give a mixture, flowing the mixture through a feed heat exchanger to heat the mixture with the heat from effluent from an ODH reactor, and adding oxygen to the mixture to give a mixed feed for the ODH reactor. The method includes dehydrogenating the ethane to ethylene via an ODH catalyst in the presence of the oxygen in the ODH reactor, and discharging the effluent from the ODH reactor, the effluent including ethylene, water, acetic acid, carbon dioxide, carbon monoxide, and unreacted ethane.

Another aspect relates to a method of producing ethylene, including dehydrogenating ethane to ethylene via an ODH catalyst in the presence of oxygen in an ODH reactor, wherein acetic acid is formed in the ODH reactor, and discharging an effluent including ethylene, acetic acid, and water from the ODH reactor. The method includes separating the effluent in a flash drum into gas and raw acetic acid, wherein the gas includes ethylene, water, acetic acid, ethane, carbon dioxide, and carbon monoxide, and wherein the raw acetic acid includes acetic acid and water. The method includes removing acetic acid and water from the gas in an acetic acid scrubber vessel and utilizing a bottoms stream discharged from the acetic acid scrubber vessel as recycle water for diluting feed to the ODH reactor.

Yet another aspect relates to an ethylene production system including an ODH reactor having an ODH catalyst to dehydrogenate ethane to ethylene and discharge an effluent including ethylene, water, acetic acid, carbon dioxide, carbon monoxide, and ethane. The ethylene production system includes a flash drum to separate effluent from the ODH reactor into gas and raw acetic acid, wherein the gas includes ethylene, water, acetic acid, carbon dioxide, carbon monoxide, and ethane, and wherein the raw acetic acid includes acetic acid and water. The ethylene production system includes an acetic acid scrubber vessel to remove acetic acid and water from the gas and discharge a bottoms stream as recycle water for diluting feed to the ODH reactor, wherein the bottoms stream includes water and acetic acid. The ethylene production system may include a cross exchanger to heat the recycle water with the effluent, and/or a cross-exchanger to receive a mixture of the recycle water and ethane to heat the mixture with the effluent. The ethylene production system may include a cross-exchanger to receive a mixture of the recycle water and oxygen to heat the mixture with the effluent for feed to the ODH reactor.

Yet another aspect relates to a method of producing ethylene, including dehydrogenating ethane to ethylene via an ODH catalyst in the presence of oxygen in an ODH reactor, discharging an effluent (including ethylene, water, acetic acid, carbon dioxide, carbon monoxide, and unreacted ethane) from the ODH reactor, recovering heat from the effluent for processing feed including ethane for the ODH reactor, recovering water from the effluent as recycle water for addition to the feed in performing water dilution of the feed, and adding oxygen to the feed to give a mixed feed including ethane and oxygen to the ODH reactor, wherein the mixed feed includes the water recovered from the effluent as the recycle water and added to the feed.

Yet another aspect relates to a method of producing ethylene, including discharging an effluent (including ethylene, water, acetic acid, carbon dioxide, carbon monoxide, and unreacted ethane) from an ODH reactor that dehydrogenates ethane to ethylene. The method includes performing water dilution of feed including ethane for the ODH reactor. The water dilution includes adding recycle water to the ethane. The method includes recovering water from the effluent to give recovered water as the recycle water for performing the water dilution. The method includes flowing the feed downstream of the water dilution through a feed heat exchanger to heat the feed with the effluent and adding oxygen to the feed to give the feed as a mixed feed for the ODH reactor.

The details of one or more implementations are set forth in the accompanying drawings and the description below. Other features and advantages will be apparent from the description and drawings, and from the claims.

### BRIEF DESCRIPTION OF DRAWINGS

FIG. 1 is a diagram illustrating an ethylene production system.

FIG. 1A is a diagram of an embodiment of the acetic acid unit of the ethylene production system of FIG. 1.

FIGS. 2-9 are diagrams of embodiments of an ethylene production system.

FIG. 10 is a plot of flammability limits as a function of the inerting concentration.

FIG. 11 is a block flow diagram of a method of producing ethylene.

Like reference numbers and designations in the various drawings indicate like elements.

### DESCRIPTION OF EMBODIMENT

Embodiments are directed to integration in an oxidative dehydrogenation (ODH) reactor system and facility of the ODH reactor feed including feed dilution. The integration includes both energy integration and water recycle. The energy integration can include providing heat from the ODH reactor effluent to heat the feed to the ODH reactor and to provide heat for diluting the feed to the ODH reactor. The diluting of the feed can include adding water (steam) diluent to the feed.

The water recycle integration in the ODH reactor system for ODH reactor feed dilution can be labeled as water recovery or system water circulation. The water recycle can include providing water recovered from the ODH reactor effluent as recovered water or recycle water for addition as water diluent to the ODH reactor feed. In some implementations, water recovered from the ODH reactor effluent to the ODH reactor feed dilution can approach a closed-circuit water system with little or no external water incorporated for diluting the feed with water.

The present disclosure includes dehydrogenating ethane to ethylene via an ODH catalyst in the presence of oxygen in the ODH reactor. Acetic acid may also be formed in the ODH reactor. The effluent discharged from the ODH reactor may include at least ethylene, acetic acid, water, carbon dioxide (CO₂), carbon monoxide (CO), and unreacted ethane. As mentioned, some aspects are directed to processing feed for the ODH reactor including to add diluent (e.g., water) to the feed.

Advantages of ODH reactor technology to produce ethylene can be lower CO₂ emissions and higher energy efficiency, as compared to steam cracking to produce ethylene. To further advance these advantages, embodiments herein may involve process integration within the ODH reactor plant to incorporate beneficial utilization of process streams in the ODH reactor system.

The feed mixture to the ODH reactor may typically include at least ethane and oxygen. To maintain the feed mixture outside of flammability conditions (outside of the flammability envelope), the feed mixture may be diluted. Steam or vaporized water can be an attractive diluent, for example, due to the relative simplicity of the separation of the water from the ODH reactor product stream (effluent) in implementations.

A significant amount of water may generally be employed to dilute the feed. Thus, the evaporating of the liquid water to generate vaporized water for addition as diluent to the feed can utilize a substantial amount of heat. In addition, cooling and condensing the water vapor (that was added to the feed) from the ODH reactor effluent can employ a significant amount of cooling capacity. These are reasons to improve the water and heat integration, as described below, between the feed dilution system and the reactor effluent cooling, acetic acid unit, and acetic acid scrubbing. These improvements in integration may reduce or eliminate need for additional water supply and reduce the load on steam and cooling tower systems.

A closed-circuit water system (or substantially closed circuit) may be implemented. In the circuit, water may be added to feed for mixed feed to the reactor, condensed along with acetic acid from reactor effluent, separated from acetic acid, recycled back to an acetic acid scrubber, and then fed to the feed dilution system. Such may reduce or generally eliminate the need for external water supply in the feed dilution.

For addition of water vapor to the hydrocarbon feed to the reactor, embodiments may employ, for example, a dilution steam drum or a saturator tower. A dilution steam drum may be more straightforward in providing dilution steam but can unfortunately rely on a higher value heat source such as medium pressure steam. In implementations, medium pressure steam can instead be better utilized, for example, to drive steam turbines. A saturator tower may saturate hydrocarbon (e.g., ethane) gas and/or oxygen gas with water vapor and utilize a relatively high circulation of water. To reduce energy demand of the dilution system (including the dilution steam drum or saturator tower), ethane and/or oxygen can also be saturated with water vapor inside heat exchangers against a heating medium such as reactor effluent or steam (e.g., low pressure steam or medium pressure steam).

This disclosure captures energy integration and water integration to provide steam diluent. Nine example options are presented below for integration to give water mixing with ethane/oxygen feed into the ODH reactor plant configuration. The nine options are given as examples. Other configurations are applicable. Options 2-9 facilitate energy consumption reduction by up to more than 40% compared to Option 1 as a base case. Option 1 may be considered a baseline case of energy integration of the hydrocarbon saturator tower into the ODH reactor plant. Although a one-stage ODH reactor (e.g., with the feed components added at the inlet) is depicted in the figures, the techniques described are applicable for other ODH reactor configurations, including multiple stage reactors and reactors with multiple inter-stage feed additions.

Embodiments along with configurations for ethane (and oxygen) feed saturation with water may be directed to process integration to cool down the reactor effluent from the ODH reactor and beneficially recover heat from the effluent. In presented options, reactor effluent that discharges from the reactor can be initially utilized to generate or superheat (very) high-pressure steam and then the effluent is cross-exchanged against reactor feed to heat the reactor feed and cool the effluent.

Embodiments include discharging an effluent including ethylene, acetic acid, and water from the ODH reactor through a steam-generation heat exchanger to generate steam and through a feed heat exchanger (cross-exchanger) to heat a feed including ethane for the ODH reactor. As mentioned, the effluent may also include CO₂, CO, and unreacted ethane. Raw acetic acid can be separated from the effluent. The raw acetic acid may be a majority of the acetic acid and water in the effluent and that is condensed. The raw acetic acid may be processed in an acetic acid unit to give acetic acid product. Gases including ethylene and unreacted ethane (and can include CO and CO₂) can be separated from the effluent and scrubbed to remove acetic acid and water to give process gas. The acetic acid and water removed can be generally the remainder of the acetic acid and water from the effluent not recovered in the raw acetic acid. In implementations, the process gas may be forwarded to a process gas compressor for further processing to give ethylene product.

As indicated, to stay outside of flammability envelope of ethane-oxygen mixture in the feed and ODH reactor, a diluent is employed. As mentioned, vaporized water or steam can be used as the diluent. Based on pressure and temperature of mixed feed including ethane, oxygen, and water to the ODH reactor, the target oxygen concentration can differ. Several process configuration schemes (e.g., including an ethane saturator tower, oxygen saturator tower, etc.) may be implemented to mix water as diluent with ethane and oxygen. Heat-integration options including different feed-saturation schemes and heating with the ODH reactor effluent are compared.

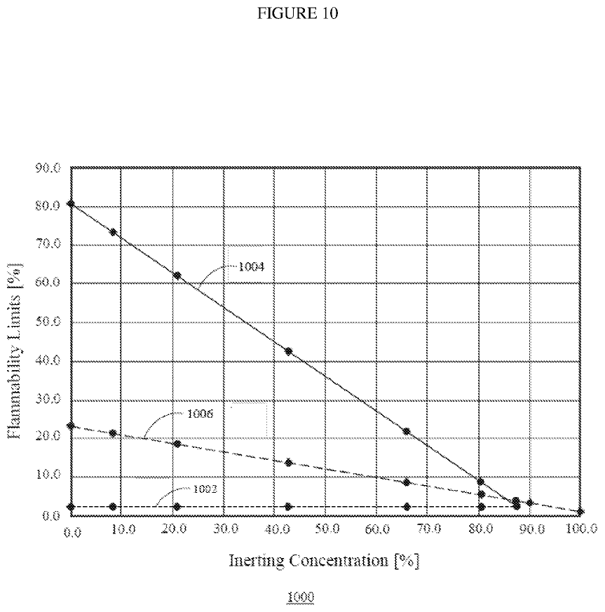

The ODH reaction to dehydrogenate feed ethane to product ethylene and generate byproduct acetic acid may occur at a temperature, for example, between 300° C. to 450° C. with low-temperature ODH catalyst (e.g., mixed metal oxides such as MoVNbTeOx and MoVTeTaOx) to produce ethylene with high selectivity. The reaction may involve feeding oxygen gas and ethane into an ODH reactor at stoichiometry ratio not less than 0.5. This corresponds to 33.3 volume percent (vol %) of oxygen gas and 66.6 vol % of ethane in the ethane-oxygen mixture. Coincidentally, such may correspond with the upper flammability limit (UFL) of ethane at 66.0 vol %, which is 25° C. and 100 kilopascal (kPa). At elevated temperature and pressure, and in order to stay outside the upper flammability limits for the mixtures, the amount of oxygen allowed in the ethane-oxygen mixture may drop (be reduced). For instance, at 300° C. and 500 kPa, the UFL of ethane in ethane-oxygen mixture is about 81 vol %. This implies that the allowed oxygen concentration in the mixture less than 19 vol %. This is indicated in FIG. 10. This amount of oxygen is less than the amount for stoichiometric reaction and results in low ethane conversion (e.g., about 40% drop in the ethane conversion at 19 vol % oxygen in the mixed). This may impose a higher load on the downstream C2 splitter due to the significant amount of unreacted ethane in the ODH reactor effluent.

A method to increase the oxygen to ethane ratio at elevated temperature and/or pressure is to add a diluent to the ethane-oxygen mixture. Examples of diluent include nitrogen, CO₂, steam, helium (He), argon (Ar), methane, etc. Based on the quenching potential of all these diluents, CO₂ appears to be the most effective with quenching factor of 1.751 at adiabatic flame temperature of 1600 K. However, unfortunately, any CO₂ used as a diluent and CO₂ generated in reaction should generally be separated from the ODH product stream. This separation may be performed, for example, with both amine and caustic towers. Due to the amount of CO₂ to be separated, the amine and caustic systems may be inefficient and relatively expensive to operate. For the case of nitrogen, He, Ar, or methane as a diluent, the diluent travels with the process gas through the downstream equipment resulting in increase of the equipment size. In addition, the utility required to separate these gases may be high, leading to high operating cost.

Steam has a quenching potential of 1.259 and may be more effective than the other diluents mentioned above except CO₂. The removal of steam from the ODH process gas by cooling before sending the product stream to the downstream equipment makes the use of steam as a diluent attractive compared to other types of diluent. However, steam may not be an inert diluent, as indicated by tests performed on fixed-bed reactor units and other information. Steam may catalyze the formation of acetic acid generation. Therefore, reducing the amount needed for dilution will reduce its impact on the amount of acetic acid generated.

Aspects herein include diluting and mixing the feed for an ODH reactor. As mentioned, several options are presented as examples.

Two major heat demands in the ODH reaction process to produce ethylene are (1) feed saturation to dilute the mixed feed; and (2) solvent recovery tower in the acetic acid (AA) unit that gives the AA product stream. Two main cooling demands for this process may be (1) reactor effluent cooling; and (2) condensing the overhead stream from the solvent recovery tower of the AA unit.

Energy integration and increasing the overall energy efficiency of the ODH reactor system including upstream feed saturation with water may decrease operating expense and emissions of greenhouse gases, such as carbon dioxide. Energy integration of reactor feed saturation, acetic acid recovery, and reactor effluent cooling is disclosed. Such may generally result not only in overall lower operating expense of the ODH reactor plant but also in lower capital expense for at least the steam system, cooling water system, and acetic acid unit.

Options of the energy integration of reactor feed saturation and reactor effluent cooling are given. The example of Option 1 presented below may be a base case. Other options presented may be generally compared to Option 1 as a baseline case. However, the present techniques are not limited to the various options as tabulated or characterized. Instead, the various options as configured including Options 1-9 are given as examples.

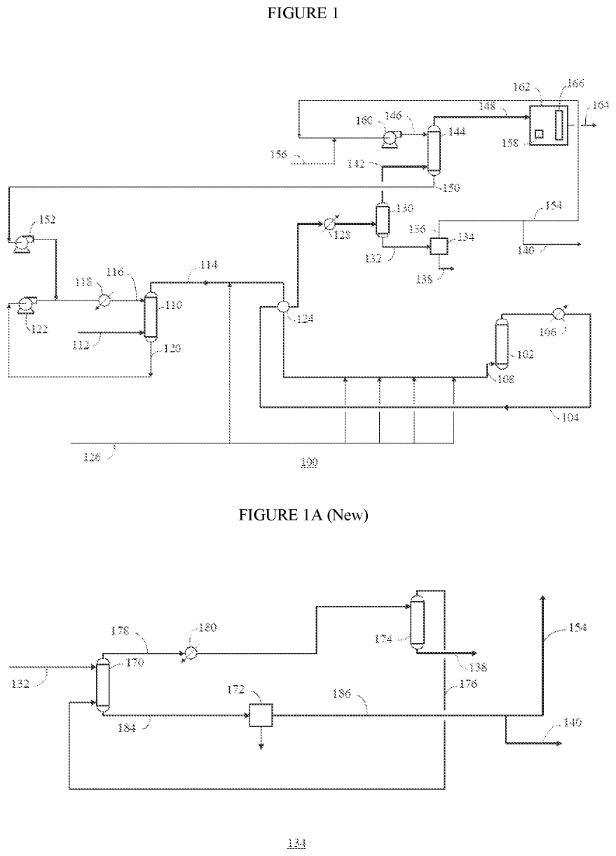

FIGS. 1-9 (Options 1-9) may be presented with respect to each other, and some include incremental differences with respect to each other. For a description of text, designations, and reference numerals depicted in a given figure of FIGS. 1-9, see also the discussion of the other figures of FIGS. 1-9. The discussion of all depicted equipment is not fully reproduced in the discussion of each figure. Instead, like reference numbers and designations in the various drawings indicate like elements.

FIG. 1 is an ethylene production system 100. FIG. 1 as depicted may be characterized as Option 1 for comparison to subsequent figures. The ethane feed saturator (ethane saturator tower) is included to give saturated ethane, which is ethane saturated in water. Oxygen gas (O₂) may be optionally added to the saturated ethane. If so added, the O₂ may be added at a single addition point or gradually in stages across multiple addition points. The saturated ethane feed may be heated (e.g., superheated) by cross exchanging with reactor effluent or other heat source. Because the amount of water (e.g., water circulation) to saturate ethane is relatively large, this FIG. 1 configuration may make possible to utilize a lower heat quality source, such as low pressure (LP) steam, at the circulation water heater for the ethane saturator tower. In other words, a large circulation of water may be implemented around the ethane saturator tower to maintain the outlet temperature of the circulation water heater relatively low so that LP steam may be advantageously utilized as the heating medium. Thus, the overall ODH reactor process may be less energy intensive as compared to configurations that utilize medium pressure (MP) steam for ethane saturation. As discussed below, the ethane feed saturator (ethane saturator tower) may be a trayed or packed bed tower.

The ethylene production system 100 includes an ODH reactor 102 vessel that has an ODH catalyst to dehydrogenate ethane to ethylene. The operating temperature of the reactor may be, for example, in the range of 300° C. to 450° C. The ODH reaction may typically be exothermic. The ODH reactor 102 system may utilize a heat-transfer fluid for controlling temperature of the reactor 102. The heat-transfer fluid may be employed to remove heat from (or add heat to) the ODH reactor 102. The heat transfer fluid can be, for example, steam, water (including pressurized or supercritical water), oil, or molten salt, and so forth. The ODH reactor 102 may be, for example, a fixed-bed reactor (operating with a fixed bed of ODH catalyst) or a fluidized-bed reactor (operating with a fluidized bed of catalyst), or another reactor type.

The ODH reaction of ethane (C₂H₆) to ethylene (C₂H₄) in the ODH reactor 102 via the ODH catalyst may include or be C₂H₆+0.5 O₂→C₂H₄+H₂O. Additional reactions in the ODH reactor 102 may include:

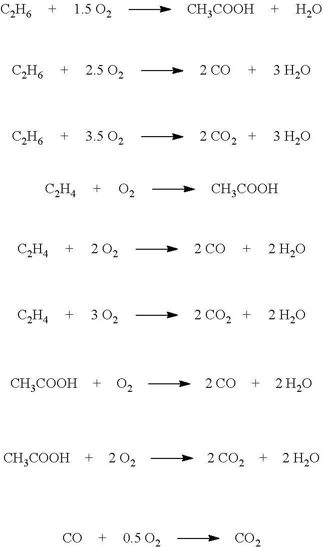

Thus, in addition the ethylene formed, water (H₂O), acetic acid (CH₃COOH), carbon monoxide (CO), and carbon dioxide (CO₂) may also be formed in the ODH reactor 102.

For the ODH reactor as a fixed-bed reactor, reactants may be introduced into the reactor at one end and flow past an immobilized catalyst. Products are formed and an effluent having the products may discharge at the other end of the reactor. The fixed-bed reactor may have one or more tubes (e.g., metal tubes, ceramic tubes, etc.) each having a bed of catalyst and for flow of reactants. For the reactor 102, the flowing reactants may be at least ethane on oxygen. The tubes may include, for example, a steel mesh. Moreover, a heat-transfer jacket adjacent the tube(s) or an external heat exchanger (e.g., feed heat exchanger or recirculation heat exchanger) may provide for temperature control of the reactor. The aforementioned heat transfer fluid may flow through the jacket or external heat exchanger.

The ODH reactor as a fluidized bed reactor can be (1) a non-circulating fluidized bed, (2) a circulating fluidized bed with regenerator, or (3) a circulating fluidized bed without regenerator. In implementations, a fluidized bed reactor may have a support for the ODH catalyst. The support may be a porous structure or distributor plate and disposed in a bottom portion of the reactor. Reactants may flow upward through the support at a velocity to fluidize the bed of ODH catalyst. The reactants (e.g., ethane, oxygen, etc. for the reactor 102) are converted to products (e.g., ethylene and acetic acid in the reactor 102) upon contact with the fluidized catalyst. An effluent having products may discharge from an upper portion of the reactor. A cooling jacket may facilitate temperature control of the reactor. The fluidized bed reactor may have heat-transfer tubes, a jacket, or an external heat exchanger (e.g., feed heat exchanger or recirculation loop heat exchanger) to facilitate temperature control of the reactor. The aforementioned heat transfer fluid may flow through the reactor tubes, jacket, or external heat exchanger.

As indicated, the ODH catalyst may be operated as a fixed bed or fluidized bed. ODH catalyst that can give an ODH reaction that dehydrogenates ethane to ethylene and forms acetic acid as a byproduct may be applicable to the present techniques. A low-temperature ODH catalyst may be beneficial. One example of an ODH catalyst that may be utilized in the ODH reactor is a low-temperature ODH catalyst that includes molybdenum, vanadium, tellurium, niobium, and oxygen, wherein the molar ratio of molybdenum to vanadium is from 1:0.12 to 1:0.49, the molar ratio of molybdenum to tellurium is from 1:0.01 to 1:0.30, the molar ratio of molybdenum to niobium is from 1:0.01 to 1:0.30, and oxygen is present at least in an amount to satisfy the valency of any present metal elements. The molar ratios of molybdenum, vanadium, tellurium, niobium can be determined by inductively coupled plasma mass spectrometry (ICP-MS). The catalyst may be low temperature in providing for the ODH reaction at less than 450° C., less than 425° C., or less than 400° C.

Associated with the ODH reaction that dehydrogenates the ethane, a byproduct formed may be acetic acid. As mentioned, also formed associated with the ODH reaction may include water, carbon dioxide, and carbon monoxide. Thus, the effluent 104 discharged from the ODH reactor 102 vessel may include ethylene, acetic acid, water, carbon dioxide, carbon monoxide, and unreacted ethane. The operating temperature of the ODH reactor 102 and the temperature of the effluent 104 as discharged may be, for example, in the range of 300° C. to 450° C.

The effluent 104 may be routed through a conduit to a steam-generation heat exchanger 106 to generate steam with heat from the effluent 104. The steam-generation heat exchanger 106 may be, for example, a shell-and-tube heat exchanger or a fin-type heat exchanger (e.g., with a finned-tube bundle), and so on. The effluent 104 may be cooled by at least 200° C. to 350° C. across the steam-generation heat exchanger 106.

Water may be heated in the steam-generation heat exchanger 106 with heat from the effluent 104 to flash the water into steam. The water may be, for example, boiler feedwater, demineralized water, or steam condensate, and the like. More than one steam-generation heat exchanger 106 may be employed in series and/or parallel. The steam generation system having the steam-generation heat exchanger 106 may include additional equipment, such as a vessel (e.g., flash vessel), a pump (e.g., boiler feedwater pump), etc. The steam generated my discharge into a steam header (or sub-header) conduit or through a conduit to a user, and so on. Higher pressure steam may generally be more valuable than lower pressure steam.

Higher pressure steam, such as greater than 500 pounds per square inch gauge (psig) or greater than 1500 psig, may typically be more valuable than lower pressure steam, such as less than 500 psig or less than 150 psig. The pressure of the steam generated via the steam-generation heat exchanger 106 may be a function of the temperature of the effluent 104 driven by the operating temperature (ODH reaction temperature) of the ODH reactor 102.

An ethane saturator tower 110 may provide water vapor for the mixed feed 108 to the ODH reactor 102 vessel. The ethane as discharged from the ethane saturator tower 110 may be water-saturated ethane. The ethylene production system 100 may include the ethane saturator tower 110 vessel (e.g., column) to incorporate water vapor into ethane gas 112 and discharge saturated ethane 114 for the mixed feed 108.

In implementations, liquid water 116 may enter an upper portion of the saturator tower 110 and flow downward through the tower 110. The tower 110 may have an inlet (e.g., nozzle) that is a flanged or screwed connection with the conduit conveying the incoming water 116. The ethane gas 112 may enter a lower portion of the saturator tower 110 and flow upward through the tower 110. The tower 110 may have packing or trays to provide contact stages of the ethane gas 112 with the water 116 for mass transfer of water vapor into the ethane gas 112. The tower 110 may include random packing, structured packing, or column trays (e.g., sieve trays, etc.), or any combinations thereof.

Liquid water 120 may discharge (e.g., as a bottoms stream) from a bottom portion of the ethane saturator tower 110 and be recirculated via a water recirculation pump 122 (e.g., centrifugal pump) as water feed to the ethane saturator tower 110. Thus, the ethane saturator tower 110 may have a water recirculation loop. The water may be heated in a circulation water heater 118 (e.g., shell-and-tube heat exchanger) with a heating medium such as steam (e.g., LP steam) to give the liquid water 116 (as heated) that enters the ethane saturator tower 110. The saturated ethane 114 may discharge overhead from the ethane saturator tower 110 for feed to the ODH reactor 102. The term “saturated” ethane as used herein means that the ethane gas is saturated with water. The saturated ethane 114 generally includes water vapor but little or no liquid water.

As discussed, the amount of water to saturate the ethane gas 112 may be relatively large. Thus, a relatively large rate of water circulated through the circulation heater 118 may be implemented to maintain the outlet temperature of the circulation water heater 118 as relatively low. Therefore, in implementations, LP steam may be advantageously utilized as the heating medium at the circulation water heater 118.

The discharged saturated ethane 114 (ethane gas saturated with water vapor) may be routed through a feed heat exchanger 124 that heats (superheats) the saturated ethane 114 as feed to the ODH reactor 102. The saturated ethane 114 may become hot at the discharge of the feed heat exchanger 124 for implementations with the ethane 114 as superheated (above water saturation temperature). The feed heat exchanger 124 may be, for example a shell-and-tube heat exchanger or a plate-fin heat exchanger. In implementations, the feed heat exchanger 124 may be a cross exchanger, as depicted, with the effluent 104 heating the saturated ethane 114. The effluent 104 may thus be cooled in the feed heat exchanger 124, e.g., typically by at least 200° C. to 350° C. In other implementations, the feed heat exchanger 124 may utilize steam instead of the effluent 104 as the heating medium.

Oxygen gas (O₂) 126 may be added to the saturated ethane 114 upstream of the feed heat exchanger 124 or downstream of the heat exchanger 124, or both. In some implementations, liquid oxygen may be received and vaporized to the vaporized O₂ as the oxygen gas. The oxygen gas 126 may be added to the saturated ethane at a single addition point or at multiple addition points (e.g., 2-5 addition points). The illustrated embodiment depicts five addition points. In certain implementations, a reason for multiple addition points may be to reduce the chance of forming a pocket of oxygen gas 126 in the flowing saturated ethane 114.

The oxygen gas 126 may be added to a conduit conveying the saturated ethane 114. In implementations, the conduit may include an in-line static mixer that is adjacent (downstream) of the addition point of the oxygen gas 126 into the saturated ethane 114. In implementations, the conduit conveying the oxygen gas 126 may tie-in to the conduit conveying the saturated ethane 114 via a pipe tee or similar pipe fitting. The mixed feed 108 to the ODH reactor 102 may include the saturated ethane 114 and the oxygen gas 126. As indicated, the water in the saturated ethane 114 may be a diluent.

The effluent 104 flows from the feed heat exchanger 124 through a cooler heat exchanger 128 to a flash drum 130. The flash drum 130 is a vessel, e.g., with a vertical orientation or horizontal orientation. In implementations, a level of liquid (e.g., raw acetic acid that may be primarily water) may be maintained in the flash drum 130 in operation.

The cooler heat exchanger 128 cools (removes heat from) the effluent 104. The cooling medium may be, for example, cooling tower water. The cooler heat exchanger 128 may be, for example, a shell-and-tube heat exchanger or plate-fin heat exchanger, or other type of heat exchanger. In implementations, the cooler heat exchanger 128 discharges the effluent 104 at a temperature, for example, in a range of 30° C. to 80° C. The cooler heat exchanger 128 may be a condenser in that water and acetic acid in the effluent 104 can condense in the cooler heat exchanger 128.

The operating pressure of the flash drum 130 may be a function of the backpressure of downstream processing of process gas (discussed below). The operating pressure of the flash drum 130 may be a function of the ODH reactor 104 discharge pressure of the effluent 104. The operating pressure of the flash drum 130 may be a function of the pressure drop associated with the flow of the effluent 104 from the ODH reactor 102 through the piping and heat exchangers to the flash drum 130 and to the downstream process gas compressor.

The temperature of the effluent 104 entering the flash drum 130 may be affected by the amount of cooling of the effluent 104 in the feed heat exchanger 124 and the cooler heat exchanger 128. The amount of water in the raw acetic acid 132 discharged as a bottoms stream from the flash drum 130 may be a function of the temperature of the effluent 104 that enters the flash drum 130. A lower temperature of the effluent 104 entering the flash drum 130 may give more water in the raw acetic acid 132. This may be so because more water will be condensed in the effluent 104 at lower temperatures. The raw acetic acid 132 may be primarily water.

In embodiments, an aspect is to cool the ODH reactor effluent 104 in the cooler heat exchanger 128 against cooling water (e.g., down to a temperature in a range of 30° C. to 80° C.) to condense a majority of the water and acetic acid in the ODH reactor effluent 104. Therefore, because a majority of water is condensed, the raw acetic acid 132 that discharges from the flash drum 130 in this embodiment may have a significant amount of water. Thus, the raw acetic acid 132 may have a low concentration of acetic acid, such as less than 1 weight percent (wt %). Depending on the embodiment and temperature of the effluent 104 entering the flash drum 130, the concentration of acetic acid in the raw acetic acid 132 can range, for example, from 0.3 wt % to 45 wt %.

The flash drum 130 discharges the raw acetic acid 132 from a bottom portion of the flash drum 130. The raw acetic acid 132 incudes liquid acetic acid and liquid water. The flash drum 130 may have outlet on the bottom portion of the flash drum 130 for the discharge of the raw acetic acid 132. The outlet may be a flanged nozzle or screwed nozzle that couples to a conduit for discharge of the raw acetic acid 132 from the flash drum 130 into the conduit. The flash drum 130 may discharge the raw acetic acid 132 through the conduit to an acetic acid unit 132, e.g., such as to an extractor column in the acetic acid unit 132.

The raw acetic acid 132 may be processed in the acetic acid unit 134 to remove water 136 from the raw acetic acid 132 to give acetic acid product 138 that is a coproduct of the ethylene production. The acetic acid product 138 may be, for example, have at least 99 wt % acetic acid. At least a portion of the water 136 removed may be recovered as water product 140. As discussed below (e.g., with respect to FIG. 1A), the acetic acid unit 134 may include an extractor column (vessel) for injection of solvent to remove acetic acid, a water stripper tower (vessel) to process raffinate from the extractor column to recover water, and a solvent recovery column (vessel) to remove the solvent from the acetic acid discharged from the extractor column to give the acetic acid product 138.

The flash drum 130 may discharge gas 142 overhead from a top portion of the flash drum 130. The gas 142 may include water vapor, residual acetic-acid vapor, and gases such as ethylene, carbon dioxide, carbon monoxide, unreacted ethane, and other gases. The other gases may include, for example, relatively small amounts of methane or propane that entered the system 100 with the ethane gas 112 (e.g., in the pipeline supply of the ethane gas 112). The flash drum 130 may include an outlet on a top portion of the flash drum 130 for discharge of the gas 142. The outlet may be a nozzle with a flange or screwed fitting to couple to a discharge conduit for discharge of the gas 142. The gas 142 may flow through the discharge conduit to an acetic acid scrubber 144, which is a vessel such as a tower or column.

A purpose of the acetic acid scrubber 144 may be to scrub (remove) acetic acid and water from the gas 142. The acetic acid and water removed from the gas 142 may generally be the remainder of acetic acid and water as sourced from the effluent 104. The removal of the acetic acid from the gas 142 may give a low concentration of acetic acid in the process gas 148 and therefore may allow for less stringent metallurgical requirements (and thus less metallurgical cost) of downstream processing equipment, such as the process gas compressor 158. The concentration of acetic acid in the process gas 148 may be, for example, less than 100 parts per million (ppm).

The scrubbing liquid may be scrubbing water 146 that enters an upper portion of the acetic acid scrubber 144 and flows downward through the acetic acid scrubber 144. The scrubber 144 may have an inlet, such as a nozzle, for receiving the scrubbing water 146. The nozzle may be, for example, a flanged or screwed connection coupled with the inlet conduit conveying the incoming scrubbing water 146. A pump 160 may provide motive force for flow of the scrubbing water 146 to the acetic acid scrubber 144. The scrubbing water 146 fed to the acetic acid scrubber 144 may include, for example, liquid water 154 from the acetic acid unit 134 and water condensate 156 from the downstream process gas compressor (PCG) 158. Condenser heat exchanger(s) at the PCG 158, including inter-stage in some examples, may condense water in the process gas 148 flowing through (being compressed in) the PCG 158.

The gas 142 from the flash drum 130 may enter a lower portion of the scrubber 144 vessel and flow upward through the scrubber 144 in a countercurrent flow with respect to the scrubbing water 146. The scrubber 144 may have an inlet (e.g., nozzle) that is a flanged or a screwed connection with the inlet conduit conveying the incoming gas 142. The acetic acid scrubber 144 may have packing or trays to provide contact stages of the gas 142 with the scrubbing water 146 for mass transfer of water vapor and acetic acid vapor from the gas 142 into the scrubbing water 146. The scrubber 144 may include random packing, ordered packing, or trays, or any combinations thereof.

The acetic acid scrubber 144 may discharge process gas 148 (e.g., overhead stream) for downstream processing to recover ethylene product. The process gas 148 may include ethylene, ethane, carbon dioxide, carbon monoxide, propane, and methane. The concentration of ethylene in the process gas 148 may be, for example, in the range of 10 mole percent (mol %) to 90 mol %. The process gas 148 is generally the gas 142 minus the acetic acid vapor and water vapor removed from the gas 142 in the scrubber 144. The process gas 148 may discharge through an outlet nozzle on a top portion of the scrubber 144, and in which the nozzle is coupled to a discharge conduit.

The scrubbing water 146 having the acetic acid vapor and water vapor removed from the gas 142 may discharge as a bottoms stream (through an outlet nozzle on a bottom portion of the scrubber 144) as recycle water 150 to the ethane saturator tower 110. The recycle water 150 may flow through a conduit to the ethane saturator 110. A recycle water pump 152 may be disposed along the conduit to provide motive force for flow of the recycle water 150. The recycle water 150 may be combined with the bottoms liquid water 120 from the ethane saturator tower 110, and flow through the circulation water heater 118 as the liquid water 116 feed to the saturator tower 110.

Provision of the recycle water 150 to the ethane saturator tower 110 may complete the circuit (e.g., closed circuit) of water recirculation in the system 100. The product water 140 discharged from the circuit may account for water generated in the ODH reaction in the ODH reactor 102. Makeup water can be added to the circuit to account for losses or process upsets. The providing of water (e.g., as the recycle water 150) recovered from the effluent 104 for mixed feed 108 dilution may give water integration in the system 100.

The process gas 148 discharged from the acetic acid scrubber 144 may be processed by downstream equipment 162 to remove ethylene from the process gas 148 as product ethylene 164. The downstream equipment 162 may include the aforementioned PCG 158 (e.g., mechanical compressor) that increases the pressure of the process gas 148. The compressed process gas may be processed to remove light components, such as carbon monoxide and methane. The downstream equipment 162 may include a C2 splitter 166 that separates ethylene from ethane. The C2 splitter 166 may be a vessel that is a distillation column having distillation trays.

In an embodiment, the ethylene production system 100 forwards the process gas 148 to the downstream equipment 162 but does not include the downstream equipment 162. Instead, the product of the ethylene production system 100 is the intermediate-product process gas 148 having ethylene. In another embodiment, the ethylene production system 100 includes the PGC 158 that discharges the process gas 148 as product. In yet another embodiment, the ethylene production system 100 includes the downstream process equipment 162. As for the downstream process equipment 162, the discussion or analysis of energy among the Options 1-9 considers the PGC 158 but typically not the remaining equipment in the downstream equipment 162.

The ODH reactor system or ODH reactor system plant may include the equipment (e.g., ethane saturator tower 110, flash drum 130, acetic acid unit 134, acetic acid scrubber 144, etc.) depicted in FIG. 1 minus the downstream process equipment 162. In some implementations, the ODH reactor system or ODH reactor system plant may be characterized as having the PGC 158 but not the splitter distillation column 166.

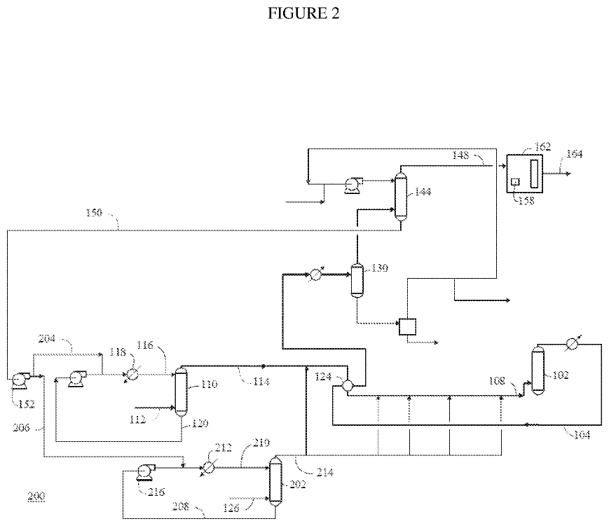

The ethylene production system 100 of FIG. 1 and the ethylene production system of subsequent FIGS. 2-9 may include a control system that facilitates or directs operation of the ethylene production system, such as the supply or discharge of flow streams (including flow rate) and associated control valves, control of operating temperatures and operating pressures, and control of columns, drums, scrubbers, and heat exchangers, and so on. The control system may include a processor and memory storing code (e.g., logic, instructions, etc.) executed by the processor to perform calculations and direct operations of the ethylene production system. The control system may be or include one or more controllers. The processor (hardware processor) may be one or more processors and each processor may have one or more cores. The hardware processor(s) may include a microprocessor, a central processing unit (CPU), a graphic processing unit (GPU), a controller card, circuit board, or other circuitry. The memory may include volatile memory (e.g., cache and random access memory), nonvolatile memory (e.g., hard drive, solid-state drive, and read-only memory), and firmware. The control system may include a desktop computer, laptop computer, computer server, programmable logic controller (PLC), distributed computing system (DSC), controllers, actuators, or control cards. Controllers may be components of the code stored in the memory and executed by the processor. The control system may include control modules and apparatuses distributed in the field.

The control system may receive user input that specifies set points of control devices or other control components in the ethylene production system. The control system typically includes a user interface for a human to enter set points and other targets or constraints to the control system. In some implementations, the control system may calculate or otherwise determine set points of control devices. The control system may be communicatively coupled to a remote computing system that performs calculations and provides direction including values for set points. In operation, the control system may facilitate processes of the ethylene production system. Again, the control system may receive user input or computer input that specifies the set points of control components in the system. The control system may determine, calculate, and specify the set point of control devices. The determination can be based at least in part on the operating conditions of the ethylene production system including feedback information from sensors and transmitters, and the like.

Some implementations may include a control room that can be a center of activity, facilitating monitoring and control of the process or facility. The control room may contain a human machine interface (HMI), which is a computer, for example, that runs specialized software to provide a user-interface for the control system. The HMI may vary by vendor and present the user with a graphical version of the remote process. There may be multiple HMI consoles or workstations, with varying degrees of access to data. The control system may also or instead employ local control (e.g., distributed controllers, local control panels, etc.) distributed in the system.

FIG. 1A is an example of the acetic acid system 134 of FIG. 1 (and subsequent figures). As discussed, the acetic acid system 134 receives raw acetic acid 132, such as from the flash drum 130. In the illustrated embodiment, the acetic acid unit 134 includes an extractor column 170 to utilize solvent to remove acetic acid from the raw acetic acid 132, a water stripper column 172 to process raffinate from the extractor column 170 to recover water, and a solvent recovery column 174 to remove the solvent from the acetic acid discharged from the extractor column 170 to give the acetic acid product 138. Again, the acetic acid unit 134 receives the raw acetic acid 132, as discussed. The raw acetic acid 132 can be primarily water.

In the illustrated implementation, the raw acetic acid 132 is fed to the extractor column 170. The raw acetic acid 132 may be introduced at an upper portion of the extractor column 170 and flow downward through the extractor column 170.

The extractor column 170 is a vessel generally having a vertical orientation. The extractor column 170 may be a liquid-liquid extraction column. The extractor column 170 may have packing (random or structured) or trays (e.g., sieve trays). If packing is employed, the packing may be metal (e.g., stainless steel) or plastic. The extractor column 170 may include moving internals (e.g., impellers) to give better contact of the liquid-liquid phases.

In operation, the extractor column 170 utilizes a solvent 176 to extract acetic acid from the raw acetic acid 132. The solvent 176 may generally be immiscible with water and thus typically does not remove a significant amount of water from the raw acetic acid 132. The solvent 176 may be, for example, n-butanol, isobutanol, amyl alcohol, ethyl acetate, or methyl tert-butyl ether (MTBE), and so forth. The solvent 176 may be introduced at a bottom portion of the extractor column 170 and flow upward through the column 170 in a countercurrent flow with the raw acetic acid 132 flowing downward through the extractor column 170. The solvent 176 removes (absorbs, extracts) acetic acid from the raw acetic acid 132. The packing or trays, and moving parts, in the extractor column 170 may facilitate mass transfer of the acetic acid into the solvent 176.

Extract 178 including the solvent 176 and the removed (absorbed, extracted) acetic acid (and can include a relatively small amount of water) from the raw acetic acid 132 discharges overhead from the extractor column 170 through an extract heater 180 (heat exchanger). The extract heater 180 heats the extract 178. The heating medium may be, for example, steam. The extract heater 180 may be a shell-and-tube heat exchanger, a plate heat exchanger, or a plate-fin heat exchanger, or other type of heat exchanger. The extract 178 is routed to the solvent recovery column 174.

The extractor column 170 discharges raffinate 184 as a bottoms stream from a bottom portion of the extractor column 170. The raffinate 184 includes the majority or bulk (e.g., nearly all) of the water from the raw acetic acid 132. The raffinate 184 is primarily water. The raffinate 184 may include trace amounts of organic compounds (e.g., solvent 176, acetic acid, etc.).

The raffinate 184 is discharged from the extractor column 170 to the water stripper column 172 to recover (increase purity of) the water. The water stripper column 172 (vessel) is a distillation column including distillation trays or packing and may be associated with a reboiler heat exchanger (or direct steam injection to the bottom) as heat source. The water stripper column 172 may be associated with an overhead condenser heat exchanger. A decanter may be employed to separate water phase from solvent phase in the condensed overheads stream from the overhead condenser. The distillation column system may include a receiver vessel or reflux drum to receive condensed liquid from the overhead condenser.

In operation, the water stripper column 172 may separate the trace amounts of organic compounds from the raffinate 184 and discharge a bottoms streams having water with the organic compounds as liquid water 186. The water stripper column 172 may discharge water vapor and organic compounds overhead that is condensed. A portion of the water 186 may be forwarded as water product 140. Another portion 154 of the water 186 may be utilized as scrubbing water 146 for the acetic acid scrubber 144.

The solvent recovery column 174 receives the extract 178 from the extract heater 180. The solvent recovery column 174 may be a distillation column that separates solvent 176 from the extract 178 to give the acetic acid product 138. The separated solvent 178 may be sent to the extractor column 170. The distillation column is a vessel having distillation trays or packing and operates with a reboiler heat exchanger and an overhead condenser heat exchanger (and a decanter to separate water-solvent phases).

The extract 178 may be introduced as a side feed (e.g., upper portion) of the solvent recovery column 174. The acetic acid product 138 may be a bottoms stream discharged from the solvent recovery column 174. The solvent 176 may be discharged overhead from the solvent recovery column 174 and then condensed.

FIG. 2 is an ethylene production system 200 that is the same or similar as the ethylene production system 100 of FIG. 1 but with the addition of an oxygen saturator tower 202 (oxygen gas saturator tower). FIG. 2 may be characterized as Option 2. For a description of equipment and reference numerals depicted in FIG. 2, see also the discussion of FIG. 1.

The oxygen saturator tower 202 and the ethane saturator tower 110 may share as feed the recycle water 150 from the acetic acid scrubber 144. A portion 204 of the recycle water 150 may be provided (via the recycle water pump 152) with the bottoms water 120 in combination as the liquid water 116 through the circulation water heater 118 to the ethane saturator tower 110. Another portion 206 of the recycle water 150 may be provided (via the recycle water pump 152) in combination with the bottoms water 208 from the oxygen saturator tower 202 as the liquid water 210 fed through the circulation water heater 212 to the oxygen (O₂) saturator tower 202.

In the illustrated embodiment with the inclusion of an oxygen saturator tower 202, the load of water incorporated into the mixed feed 108 (for saturating or saturation) is shared between the two saturator towers 110, 202. This may also reduce the water circulation rate and quality of the LP steam demand at the circulation water heaters 118, 212, and lower the temperature of the circulating water discharged from the circulation water heaters 118, 212, as compared with circulation water heater 118 in Option 1. See, for instance, Table 1 in which LP steam at lower-pressure 60 psig is implemented for Option 2 as compared to LP steam at the higher-pressure 70 psig. This gives a lower temperature of the circulating water for Option 2 as compared to Option 1. The oxygen saturator tower 202 (oxygen feed saturator) can be a trayed tower or packed bed tower.

The oxygen saturator tower 202 may receive the oxygen gas 126 (supply) and provide saturated oxygen gas 214 (saturated with water) for the mixed feed 108 to the ODH reactor 102 vessel. The ethylene production system 100 may include the oxygen saturator tower 202 vessel (e.g., column) to incorporate water vapor into the oxygen gas 126 and discharge saturated oxygen gas 214 for the mixed feed 108.

In implementations, liquid water 210 may enter an upper portion of the saturator tower 202 and flow downward through the tower 202. The tower 202 may have an inlet (e.g., nozzle) that is a flanged or screwed connection with the conduit conveying the incoming water 210. The oxygen gas 126 may enter a lower portion of the saturator tower 202 and flow upward through the tower 202. The tower 202 may have packing or trays to provide contact stages of the oxygen gas 126 with the water 210 for mass transfer of water vapor into the oxygen gas 126. The tower 202 may include random packing, structured packing, or column trays (e.g., sieve trays, etc.), or any combinations thereof.

Liquid water 208 may discharge (e.g., as a bottoms stream) from a bottom portion of the oxygen saturator tower 202 and circulate (recirculate) via a water circulation (recirculation) pump 216 (e.g., centrifugal pump) as water feed to the oxygen saturator tower 202. Thus, the oxygen saturator tower 202 may have a water recirculation loop. As mentioned, the bottoms liquid water 208 may be combined with the portion 206 of the recycle water 150 to give the liquid water 210 introduced into an upper portion of the oxygen saturator tower 202.

The feed liquid water 210 may be heated in the circulation water heater 212 with a heating medium such as steam (e.g., LP steam) to give the liquid water 210 (as heated) that enters the oxygen saturator tower 202. The circulation water heater 212 is a heat exchanger, such as a shell-and-tube heat exchanger or plate-fin heat exchanger, and the like. The saturated oxygen gas 214 may discharge overhead from the oxygen saturator tower 202 for feed to the ODH reactor 102. The “saturated” oxygen or “saturated” oxygen gas means that the oxygen gas is saturated with water (water vapor) and thus at water saturation. The saturated oxygen gas 214 includes water vapor but little or no liquid water.

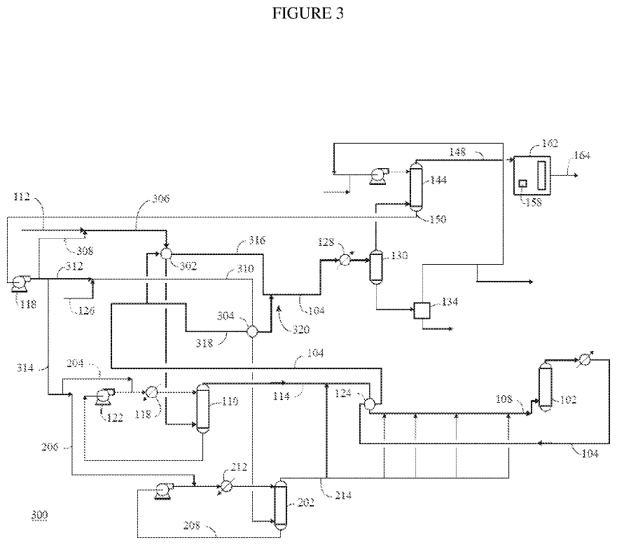

FIG. 3 is an ethylene production system 300 that is the same or similar as the ethylene production system 200 of FIG. 2 but with an ethane cross-exchanger 302 and an oxygen cross-exchanger 304, and associated water addition for partial saturation. FIG. 3 may be labeled as Option 3. For a description of text, designations, and reference numerals depicted in FIG. 3, see also the discussion of FIGS. 1 and 2.

Inclusion of the ethane cross-exchanger 302 and an oxygen cross-exchanger 304 may beneficially lead to less LP steam demand at the respective circulation water heaters 118, 212 of the saturator towers 110, 202 due to significant heat recovery from reactor effluent 104. Plant cooling water demand may also be reduced (e.g., reduced load on the cooling tower) due to heat recovery from (cooling of) the reactor effluent via the cross-exchangers 302, 304. The cross-exchangers 302, 304 may each be a heat exchanger, such as plate (and fin) heat exchanger or a shell-and-tube heat exchanger with heat source and heat sink on either side.

In operation, the ethane cross-exchanger 302 heats a mixture 306 of the ethane gas 112 and the recycle water 308. The recycle water 308 may be a portion of the recycle water 150 from the bottoms of the acetic acid scrubber 144. The mixture 306 (as heated) downstream of the ethane cross-exchanger 302 may be labeled as partially-saturated ethane that is fed to the ethane saturator 110. Thus, instead of feeding the ethane gas 112 directly to the ethane saturator tower 110 as in FIGS. 1 and 2, the ethane gas 112 is first partially saturated with the recycle water 308 prior to introduction to the ethane saturator tower 110.

The oxygen cross-exchanger 304 heats a mixture 310 of the oxygen gas 126 and the recycle water 312. The recycle water 312 may be another portion of the recycle water 150 from the bottoms of the acetic acid scrubber 144. The remainder 314 of the recycle water 150 may be fed to the ethane saturator 110 and the oxygen saturator tower 202 (as portions 204 and 206, respectively), as depicted. The mixture 310 (as heated) downstream of the oxygen cross-exchanger may be labeled as partially-saturated oxygen that is fed to the oxygen saturator tower 202 for saturation of the oxygen gas with water. Thus, instead of feeding the oxygen gas 126 directly to the oxygen saturator tower 202 as in FIG. 2, the oxygen gas 126 is first partially saturated with the recycle water 312 prior to introduction to the oxygen saturator tower 202.

The ethane cross-exchanger 302 and the oxygen cross-exchanger 304 may each be a shell-and-tube heat exchanger, plate heat exchanger, or a plate-fin heat exchanger, or other type of heat exchanger. The ethane cross-exchanger 302 and the oxygen cross-exchanger 304 may utilize the effluent 104 as the heating medium, either in series or in parallel as depicted.

In the illustrated implementation, the ethane cross-exchanger 302 and the oxygen cross-exchanger 304 receive the effluent 104 downstream of the feed heat exchanger 124. A portion 316 of the effluent 104 is fed to the ethane cross-exchanger 302. The remaining portion 318 of the effluent 104 is fed to the oxygen cross-exchanger 304. The effluent 104 may be divided as portions 316 and 318, for example, via a pipe tee or other piping fitting. Thus, the conduit conveying the effluent 104 may discharge to two conduits conveying the portions 316 and 318, respectively. A control valve (e.g., flow control valve) may be disposed on one of the two conduits. Other arrangements or configurations for dividing the effluent 104 into the portions 316 and 318 are applicable.

Downstream, the portions 316 and 318 of the effluent 104 may be combined to give the effluent 104 going forward as cooled by the ethane cross-exchanger 302 and the oxygen cross-exchanger 304. The portions 316 and 318 may be combined (as indicated at arrow 320) upstream of the cooler heat exchanger 128. The effluent 104 (as cooled) may flow through the cooler heat exchanger 128 (for additional cooling) to the flash drum 130.

Incorporation of the two parallel cross-exchangers 302 and 304 provides for cooling the effluent and therefore reduces cooling water demand for cooling the effluent 104 (e.g., reduces demand of cooling tower water at the cooler heat exchanger 128) as compared to Options 1 and 2. Furthermore, the addition of the two parallel cross-exchangers 302 and 304 recovers heat from the effluent 104 for feed saturation (e.g., for water saturation of the ethane gas 112 and oxygen gas 126, and thus for diluting the mixed feed 108 with water). Therefore, steam consumption (e.g., LP steam at the circulation water heaters 118, 212) for feed saturation may be reduced as compared with Option 2. However, the addition of the two parallel cross-exchangers 302 and 304 between the ODH reactor 102 and PGC 158 may result in a lower suction pressure for PGC 158 and thus higher PGC 158 power consumption as compared to Options 1 and 2.

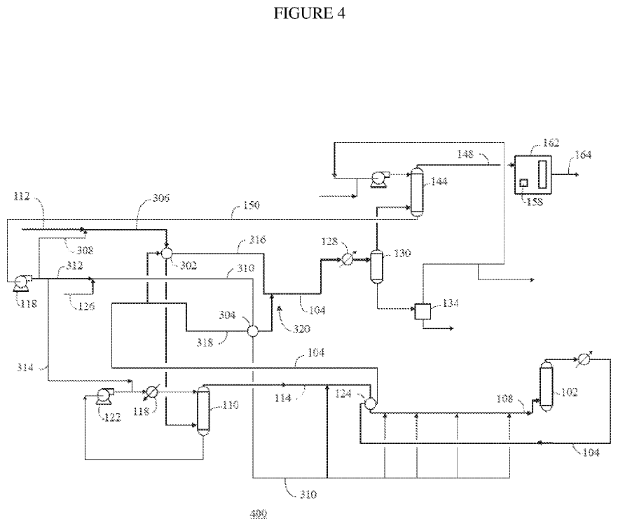

FIG. 4 is an ethylene production system 400 that is the same or similar as the ethylene production system 300 of FIG. 3 but without the oxygen saturator tower 202. FIG. 4 may be labeled as Option 4. For a description of text, designations, and reference numerals depicted in FIG. 4, see also the discussion of the preceding figures.

Elimination of the oxygen (O₂) saturator tower 202 might simplify the ethylene production system compared to Option 3. However, the quality of LP steam demand at the circulation water heater 118 for the ethane saturator tower 110 may be higher than Options 2 and 3.

The remainder 314 of the recycle water 150 may be fed to the ethane saturator 110. The remainder 314 of the recycle water 150 may be equivalent to portion 204 (FIGS. 2-3) without a portion 206 utilized for oxygen gas saturation or an oxygen saturator tower 202.

In FIG. 4, the oxygen cross-exchanger 304 heats the mixture 310 of the oxygen gas 126 and the recycle water 312, as in FIG. 3. However, in FIG. 4, the heated mixture 310 (partially saturated oxygen) is added to the conduit conveying the saturated ethane 114 (instead of fed to an oxygen saturator tower 202 as in FIG. 3).

Moreover, in comparison to FIG. 1, instead of adding the oxygen gas 126 directly to the saturated ethane 114 as in FIG. 1, the oxygen gas 126 in FIG. 4 is first partially saturated with the recycle water 312 via the oxygen cross-exchanger 304 prior to introduction to the conduit conveying the saturated ethane 114.

The heated mixture 310 (partially saturated oxygen) may be added to the saturated ethane 114 at a single addition point or at multiple addition points (e.g., 2-5 addition points). The heated mixture 310 may be added to the saturated ethane 114 upstream or downstream of the feed heat exchanger 124, or both. The combination of the saturated ethane 114 and the mixture 310 (as heated by the cross-exchanger 304) may be the mixed feed 108 introduced to the ODH reactor 102 vessel.

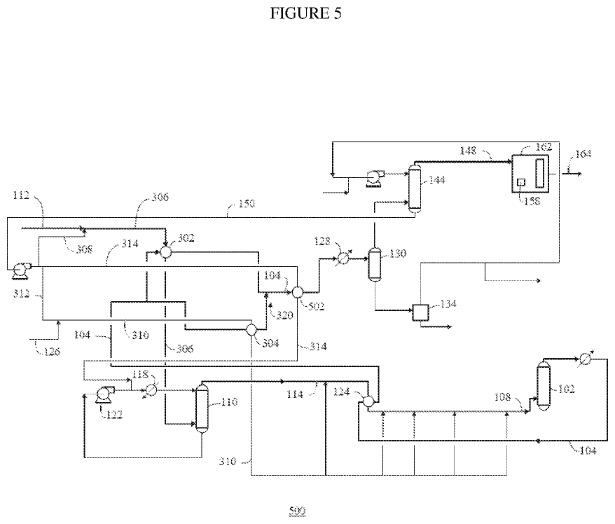

FIG. 5 is an ethylene production system 500 that is the same or similar as the ethylene production system 400 of FIG. 4 but with the addition of a recycle water cross-exchanger 502 to heat the remainder 314 of the recycle water 150 with reactor effluent 104. FIG. 5 may be labeled as Option 5. For a description of text, designations, and reference numerals depicted in FIG. 5, see also the discussion of the preceding figures.

The recycle water cross-exchanger 502 heats (with effluent 104 as the heating medium) the remainder 314 portion of the recycle water 150 fed to the ethane saturator tower 110. Thus, the recycle water cross-exchanger 502 preheats recycle water to the ethane saturator tower 110 (ethane feed saturator). The recycle water cross-exchanger 502 further increases heat recovery from reactor effluent 104. Utilization of the recycle water cross-exchanger 502 reduces both the steam demand for feed saturation and the cooling water demand for reactor effluent 104 cooling compared to Option 4. As with the cross-exchangers 302, 304, the recycle water cross-exchanger 502 can be, for example, a plate (and fin) heat exchanger or a shell-and-tube heat exchanger with the heat sink and heat source being on either side, respectively. In the illustrated embodiment, the recycle water cross-exchanger 502 is operationally disposed along the effluent 104 flow downstream of the cross-exchangers 302, 304, and upstream of the cooler heat exchanger 128.

As discussed, the recycle water 150 is the bottom streams discharged from the acetic acid scrubber 144. Portions 308 and 312 of the recycle water 150 are taken for partially saturating the ethane gas 112 and oxygen gas 126, respectively, as is done in FIG. 4. However, the remaining recycle water 150 is routed as a remainder portion 314 through the recycle water cross-exchanger 502 in route to (before being sent through) the circulation water heater 118 to the ethane saturator tower 110.

As indicated, the cross-exchangers 302, 304, and 502 may be a shell-and-tube heat exchanger, plate heat exchanger, or a plate-fin heat exchanger, or other type of heat exchanger. Further, as generally for the cross-exchangers discussed herein, the system 500 may be configured for routing the heating medium and cooling medium through either side of the cross-exchanger, respectively. For instance, the cross-exchanger as a shell-and-tube heat exchanger may be configured such that the heating medium (effluent 104 for cross-exchanger 502) flows through the tubes (tube bundle) and the cooling medium (remainder 314 of recycle water for cross-exchanger 502) flows through the shell. Alternatively, the cross-exchanger may be configured such that the heating medium flows through the tubes and the cooling medium flows through the shell.

As also indicated, employing the recycle water cross-exchanger 502 may further reduce steam consumption (e.g., LP steam at the circulation water heater 118) for ethane saturation as compared with FIG. 4 (Option 4). The addition of the recycle water cross-exchanger 502 may also further reduced cooling water demand at the cooler heat exchanger 128 for cooling the effluent 104 as compared to Option 4. Yet, the addition of another heat exchanger (recycle water cross-exchanger 502) in which the effluent 104 flows through may increase pressure drop between the ODH reactor 102 and the PGC 158, which could lead to more power demand by the PGC 158.

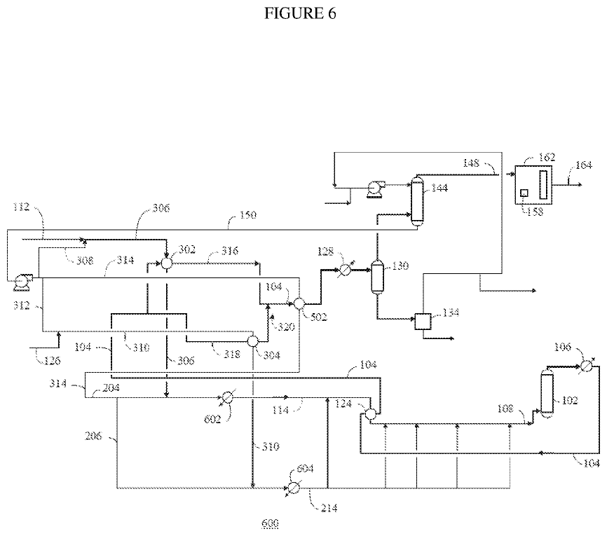

FIG. 6 is an ethylene production system 600 that is the same or similar as the ethylene production system 500 of FIG. 5 but with inclusion of an ethane saturator heat exchanger 602 and an oxygen (O₂) saturator heat exchanger 604. The ethane saturator heat exchanger 602 replaces the ethane saturator tower 110, as compared to FIG. 5. FIG. 6 may be characterized as Option 6. For a description of text, designations, and reference numerals depicted in FIG. 6, see also the discussion of the preceding figures.

The ethane saturator heat exchanger 602 and the oxygen saturator heat exchanger 604 may each be, for example, a plate (and fin) heat exchanger or a shell-and-tube heat exchanger. The heat sink (process stream here) and the heat source (utility heating medium) may flow through either side, respectively, of the heat exchanger. The process side and the utility side may be either side of the heat exchanger.

For the ethane saturator heat exchanger 602, the heat sink (heated mixture 306 combined with portion 204 of recycle water) and the heat source (e.g., LP steam) may flow through either side, respectively, of the ethane saturator heat exchanger 602. For the oxygen saturator heat exchanger 604, the heat sink (heated mixture 310 combined with the portion 206 of recycle water) and the heat source (e.g., LP steam) may flow through either side, respectively, of the oxygen saturator heat exchanger 604.

The ethane saturator heat exchanger 602 replaces the ethane saturator tower 110 and associated water circulation pump 122 and circulation water heater 118. The ethane saturator heat exchanger 602 is employed instead of the ethane saturator tower 110 to further heat and saturate with water (recycle water) the heated mixture 306 that is ethane gas partially saturated with water (recycle water). Therefore, the ethane saturator tower 110 system including the tower 110, water circulation pump 122, and the circulation water heater 118 may be eliminated. Thus, Option 6 may be more straightforward and power efficient compared to Option 5.

As discussed, the main incoming flow of recycle water for saturating feed may be the overall recycle water 150 that is the bottom streams discharged from the acetic acid scrubber 144. In FIG. 6, portions 308 and 312 of the recycle water 150 are taken for partially saturating the ethane gas 112 and oxygen gas 126, respectively, as is done in FIGS. 4 and 5. The remainder 314 of the recycle water 150 is flowed through the recycle water cross-exchanger 502 as in FIG. 5 but in FIG. 6 the heated remainder 314 is provided as recycle water to the ethane saturator heat exchanger 602 and the oxygen saturator heat exchanger 604. In particular, the portion 204 of the remainder 314 of recycle water 150 is provided to the ethane saturator heat exchanger 602. The portion 206 of the remainder 314 is provided to the oxygen saturator heat exchanger 604.

The portion 204 is combined with heated mixture 306 (ethane gas partially saturated with water) routed through the ethane saturator heat exchanger 602 to give the saturated ethane 114. The ethane saturator heat exchanger 602 utilizes a heating medium (e.g. LP steam) to heat and vaporize the liquid-water portion 206 into water vapor for saturating (e.g., fully saturating) the heated mixture 306 with water to discharge the saturated ethane 114. As in previous figures, saturated ethane 114 is routed through the feed heat exchanger 124 and enters in the mixed feed 108 to the ODH reactor 102.

The portion 206 of the remainder 314 of the recycle water 150 is combined with the heated mixture 310 (oxygen gas partially saturated with water) in route through the oxygen saturator heat exchanger 604. This combined flow as heated (e.g., via LP steam) in the oxygen saturator heat exchanger 604 discharges from the heat exchanger as saturated oxygen 214. This saturated oxygen 214 may be the same or similar as the saturated oxygen 214 that discharges overhead from the oxygen saturator tower 202 in FIGS. 2-3. In FIG. 6, an oxygen saturator tower 202 is not employed. The saturated oxygen 214 is oxygen gas saturated (e.g., fully saturated) with water vapor. As in FIGS. 2-3, saturated oxygen 214 is added to the saturated ethane 114 and is introduced in the mixed feed 108 to the ODH reactor 102.

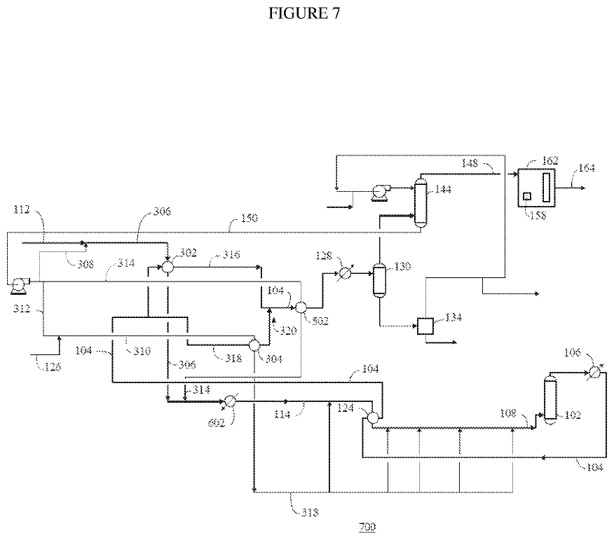

FIG. 7 is an ethylene production system 700 that is the same or similar as the ethylene production system 600 of FIG. 6 but without the oxygen saturator heat exchanger 604. FIG. 7 may be characterized as Option 7. For a description of text, designations, and reference numerals depicted in FIG. 7, see also the discussion of the preceding figures. Option 7 is not much different than Option 6 but may be considered slightly more straightforward in not having the oxygen saturator heat exchanger 604. The heating demand of Option 7 versus Option 6 on the supply steam system at the facility may be similar.

As indicated, the ethylene production system 700 does not include the oxygen saturator heat exchanger 604 (of FIG. 6) that fully saturates the oxygen feed for addition to the saturated ethane 114. The system 700 also does not include an oxygen saturator tower 202 (FIGS. 2-3). Thus, the heated mixture 310 (oxygen gas 126 partially saturated with water) that discharges from the oxygen cross-exchanger 304 is added to the saturated ethane 114 for the mixed feed 108, as discussed with respect to FIG. 4.

The remainder 314 of the recycle water 150 heated in the recycle water cross-exchanger 502 (as in FIG. 5 and FIG. 6) is provided to the ethane saturator heat exchanger 602 for fully saturating the mixture 306 (ethane gas 112 partially saturated with water) to give the saturated ethane 114. The remainder 314 of the recycle water 150 is not divided into portions 204 and 206 as in FIG. 6 because there is no oxygen saturator heat exchanger 604 in FIG. 7.

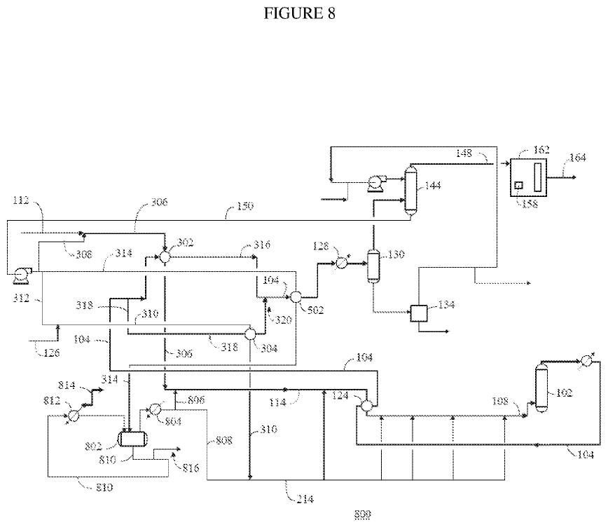

FIG. 8 is an ethylene production system 800 that is the same or similar as the ethylene production system 700 of FIG. 7 but with the inclusion of a dilution steam drum 802 and without the ethylene saturator heat exchanger 602. FIG. 8 may be labeled as Option 8. For a description of text, designations, and reference numerals depicted in FIG. 8, see also the discussion of the preceding figures.

The dilution steam (DS) drum 802 provides dilution steam for fully saturating both the ethane and O₂ with water. In particular, the DS drum 802 provides dilution steam for addition to the heated mixture 306 (partially saturated ethane) to give the saturated ethane 114. The DS drum 802 provides dilution steam for addition to the heated mixture 310 (partially saturated oxygen gas) to give the saturated oxygen gas 214. As discussed, the saturated oxygen gas 214 is added to the saturated ethane 114 to give the mixed feed 108 fed to the ODH reactor 102.

In FIG. 8, as described with respect to FIGS. 4-7, portions 308 and 312 of the recycle water are utilized for partially saturating the ethane gas 112 and oxygen gas 126 in the ethane cross-exchanger 302 and oxygen cross-exchanger 304, respectively. In FIG. 8, the remainder 314 of the recycle water 150 is sent through the recycle water cross-exchanger 502 to the DS drum 802. Thus, heat is recovered from the reactor effluent 104 to the DS drum 802 via heating of the remainder 314 of the recycle water 150 in the recycle water cross-exchanger 502.

The DS drum 802 is a vessel that may have a horizontal orientation or a vertical orientation. The DS drum 802 may have a vessel inlet nozzle to receive the remainder 314 of the recycle water 150 into DS drum 802 vessel. In operation, a liquid level of water is maintained in the DS drum 802, such as via liquid level controls. The liquid level controls may include a level control valve on a discharge conduit from a bottom portion of the DS drum 802, a level sensor at the DS drum 802 vessel, an instrument transmitter to indicate the liquid level as measured by the level sensor to a control system, and control logic in the control system to control the level control valve to maintain the liquid level at a set point. The operating temperature in the DS drum 802 may be, for example, in the range of 135° C. to 175° C.

In operation, liquid water flashes (vaporizes) in the DS drum 802 and discharges overhead as steam from the DS drum 802. Any water vapor (steam) that enters the DS drum 802 in the circulated (recirculated) water 810 also discharges overhead in the discharged steam stream. The steam that discharges overhead from the DS drum 802 is dilution steam and may be saturated steam (or slightly above saturation temperature). The DS drum 802 may have a vessel outlet nozzle on an upper or top portion of the DS drum 802 for discharge of the saturated dilution steam. The dilution steam (e.g., saturated dilution steam or near saturation) may discharge into a conduit for flow through a superheater 804.

The dilution steam may flow through a superheater 804 prior to use of the dilution steam as a diluent. The superheater 804 may be a heat exchanger, such as a shell-and-tube heat exchanger or a plate-fin type heat exchanger. The superheater 804 utilizes steam (e.g., MP steam) as a heating medium to heat the dilution steam to above saturation temperature to give the dilution steam as superheated steam.

In the illustrated embodiment, the superheated dilution steam may be divided (split) into two portions 806 and 808 into two respective conduits. The portion 806 of the superheated dilution steam is added to the heated mixture 306 (partially saturated ethane) to give the saturated ethane 114. For example, the portion 806 of the superheated dilution steam may flow through a conduit downstream of the superheater 804 and added (e.g., via a pipe tee or other pipe fitting) to a conduit conveying the heated mixture 306 from the ethane cross-exchanger 302.

The portion 808 of the superheated dilution steam is added to the heated mixture 310 (partially saturated oxygen gas) to give the saturated oxygen gas 214. For example, the portion 808 of the superheated dilution steam may flow through a conduit downstream of the superheater 804 and added (e.g., via a pipe tee or other pipe fitting) to a conduit conveying the heated mixture 310 from the ethane cross-exchanger 304.

Liquid water 810 may discharge from a bottom portion of the DS drum 802 into a discharge conduit. The aforementioned level control valve may be disposed along the discharge conduit. As discussed below, the liquid water 810 may circulate (e.g., via thermosiphon, pump, etc.) back to the DS drum 802.

In some implementations, the liquid water 810 (or a slipstream of the liquid water 810) as discharged from the DS drum 802 may be sent to blowdown (e.g., sewer), as indicated by reference numeral 816, to prevent or reduce accumulation of impurities (e.g., solids) in the DS drum 802. The blowdown may be intermittent.

The liquid water 810 may circulate (e.g., via thermosiphon) back to the DS drum 802 and be heated in a DS generator 812 in the circulation loop. Vaporization of water 810 may occur in the circulation loop including in the DS generator 812. Thus, flow of water 810 through in the circulation loop may be two-phase flow of liquid water and steam (water vapor). The water 810 may flow through the DS generator 812 back to the DS drum 802. The DS generator 812 may be a heat exchanger, such as a shell-and-tube heat exchanger or a plate-fin type heat exchanger. The heating medium may be, for example, MP steam 814. In some embodiments, the MP steam 814 may be injected into the circulating water 810 at the DS generator 814 in addition to being a utility side fluid of a heat exchanger.

The DS drum 802 may have an inlet nozzle to receive the circulating liquid water 810 through a conduit from the DS generator 812. There may be partial steam generation in the DS generator 812. As mentioned, in certain implementations, some vaporization of the water 810 can occur in the circulation piping and the DS generator 812. Therefore, the circulating liquid water 810 can include water vapor including downstream of the DS generator 812 and thus be two-phase flow.

The configuration for dilution steam as in Option 8 may be a straightforward technique to add water (for dilution) to the feed (ethane and oxygen) to give the mixed feed 108. However, this technique in Option 8 may utilize a higher value steam than Options 1-7 for the feed saturation. For instance, Option 8 may employ MP steam 814 (at the DS generator 812) compared to previous options employing LP steam, for example, at the circulation water heater 118 for the ethane saturator tower. Yet, Option 8 may beneficially recover significant heat from the reactor effluent 104. Option 8 can be a better option with respect to energy as compared to Option 1 and 2 in implementations.

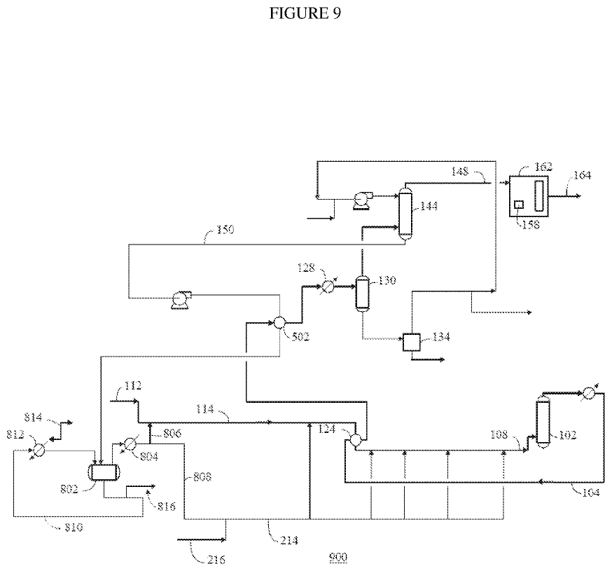

FIG. 9 is an ethylene production system 900 that is the same or similar as the ethylene production system 800 of FIG. 8 but without the ethane cross-exchanger 302 and oxygen cross-exchanger 304. FIG. 9 may be labeled as Option 9. For a description of text, designations, and reference numerals depicted in FIG. 9, see also the discussion of the preceding figures.

The portion 806 of the superheated dilution steam is added to the ethane gas 112 to give the saturated ethane 114. The portion 806 may be transported via a conduit and added, for example, to a conduit conveying the ethane gas 112. A pipe fitting (e.g., pipe tee) may be employed for tie-in of the two conduits. The flow rate of the portion 806 in Option 9 may be greater relative to the flow rate of the portion 806 in Option 8 because Option 9 does not employ the ethane cross-exchanger 302 in which recycle water is incorporated into the ethane gas for dilution giving partial saturation.

The portion 808 of the superheated dilution steam is added to the oxygen gas 126 to give the saturated oxygen gas 214. The portion 808 may be transported via a conduit and added, for example, to a conduit conveying the oxygen gas 126. A pipe fitting (e.g., pipe tee) may be employed for tie-in of the two conduits. As discussed, the saturated oxygen gas 214 is added to the saturated ethane 114 to give the mixed feed 108 fed to the ODH reactor 102. The flow rate of the portion 808 in Option 9 may be greater relative to the flow rate of the portion 808 in Option 8 because Option 9 does not employ the oxygen cross-exchanger 304 in which recycle water is incorporated into the oxygen gas for dilution giving partial saturation.

The recycle water 150 (e.g., as a bottoms stream from the acetic acid scrubber 144) is fed through the recycle water cross-exchanger 502 to the DS drum 802. The recycle water cross-exchanger 502 heats the recycle water 150 with the reactor effluent 104 as the heating medium. Thus, heat is beneficially recovered from the effluent 104 to the DS drum 802.

Option 9 employs a straightforward approach to dilute the mixed feed 108 with water. However, utilization of MP steam in implementation may offset the heat recovery from reactor effluent 104 for energy integration as compared to Option 1.

As can be appreciated, the vessels and heat exchangers discussed with respect to FIGS. 1-9 may have at least one inlet (e.g., nozzle) that is a flanged or screwed connection with an inlet conduit, and at least one outlet (e.g., nozzle) that is a flanged or screwed connection with an outlet conduit.

More than one ODH reactor 102 may be employed, including in series and/or parallel. Although the ODH reactor 102 is depicted as a or one-stage reactor, e.g., with all feed components (mixed feed 108) added at the inlet of the reactor, the processes described are applicable for other reactor configurations, including multiple stage reactors and reactors with multiple inter-stage feed additions.

The steam generated or utilized may be low pressure (LP) steam (e.g., 150 psig or less), medium pressure (MP) steam (e.g., in the range of 150 psig to 500 psig), high pressure (HP) steam (e.g., 500 psig or greater), or very high pressure (VHP) steam (e.g., 1500 psig or greater), and so forth. Again, at the steam generator 106, generation of HP steam or VHP steam may be generally more valuable than generating MP steam or LP steam and thus improve economics of the ethylene production system 100. There may be different applications for the steam. The use of the steam by the consumers or customers receiving the steam may depend on the pressure or quality of the steam. In some implementations, higher steam pressures of the produced steam may give more versatility in the integration of the steam within the facility or plant. For instance, HP steam can be utilized to power turbines attached to compressors, while LP steam is typically used for heating purposes, and the like. As discussed for some of the feed dilution embodiments, MP steam may be employed which is a higher value steam than LP steam. The source of LP steam and MP steam can include, for example, extraction turbines or depressurizing valve for HP or VHP steam.

As indicated, the ODH reactor 102 may be a fixed-bed reactor (e.g., a tubular fixed-bed reactor), a fluidized-bed reactor, an ebullated bed reactor, or a heat-exchanger type reactor, and so on. A fixed-bed reactor may have a cylindrical tube(s) filled with catalyst pellets as a bed of catalyst. In operation, reactants flow through the bed and are converted into products. The catalyst in the reactor may be one large bed, several horizontal beds, several parallel packed tubes, or multiple beds in their own shells, and so on.

A fluidized bed reactor may be a vessel in which a fluid is passed through a solid granular catalyst (e.g., shaped as spheres or particles) at adequate velocity to suspend the solid catalyst and cause the solid catalyst to behave as though a fluid. In implementations, a fluidized bed reactor may have a support for the catalyst. The support may be a porous structure or distributor plate and disposed in a bottom portion of the reactor. Reactants may flow upward through the support at a velocity to fluidize the bed of catalyst (e.g., the catalyst rises and begins to swirl around in a fluidized manner). A fluidized bed reactor has a recirculating mode of operation.

The techniques may include maintaining an operating temperature of the ODH reactor 102 at less than 450° C., less than 425° C., or less than 400° C. As for operating pressure, the reactor 102 inlet pressure may be less than 80 pound per square inch gauge (psig), or less than 70 psig. The reactor inlet pressure for each reactor may in the range of 1 psig to 80 psig, or in the range of 5 psig to 75 psig. Other operating conditions of the reactor 102 in the embodiments of the reactor 102 as a tubular fixed-bed reactor may be gas hourly space velocity (GHSV) in the range of 200 hour⁻¹ to 25,000 hour⁻¹ and a linear velocity range of the feed through the reactor of at least 5 centimeters per second (cm/sec).

FIG. 10 is a flammability diagram 1000 for a mixture of ethane gas, oxygen gas (O₂), and water vapor (steam). The water is diluent as the inerting component. The flammability diagram 1000 is a plot of flammability limits in mole percent (mol %) of O₂ in the mixture versus inerting concentration that is the mol % of water in the mixture. The curve (line) 1002 is the lower flammability limit (mol % O₂) of the mixture as a function of the O₂ concentration (mol % O₂) over the inerting concentration (mol % water). The curve (line) 1004 is the upper flammability limit of the mixture as a function of the O₂ concentration over the inerting concentration. The region between the curve 1002 and the curve 1004 may be labeled as the flammability zone (flammability envelop) of the mixture of ethane, O₂, and water. The curve (dashed line) 1006 is the O₂ concentration at the stoichiometric relationship with the ethane concentration over the inerting concentration. Lastly, as indicated by the diagram 1000, the flammability envelop narrows at a temperature and a pressure with the inclusion of diluent (e.g., water). As discussed, water may be added to the feed to the ODH reactor to narrow the flammability envelope of the feed (and maintain the feed outside of the flammability envelope) in the feed line and in the ODH reactor. In particular, the inclusion of water adjusts the concentration of ethane and oxygen to take the mixture outside of the flammability envelope.

Below are comments regarding implementations of Options 1-9 and similar options. First, the techniques may target, for example, between 0.5 mol % to 6 mol % less O₂ concentration than the limiting oxygen concentration (LOC). In other words, the target O₂ mol % in the mixed feed to the ODH reactor may be, for example, the LOC minus a value (e.g., in the range of 0.5 mol % to 6 mol %) at a given temperature and pressure in the mixed feed. This may facilitate that the feed remains outside of the flammability zone from the beginning of mixing of oxygen gas with the ethane in the feed conduit until the mixed feed is introduced to the ODH reactor.

Second, the addition of the O₂ gradually (in other words, over multiple addition points) to the ODH reactor feed (which includes ethane) in the conduit conveying the feed may reduce the chance or probability of the feed suddenly and temporarily entering the flammable zone.

Third, a closed circulation (or substantially closed circulation) of water may be utilized to supply the water for dilution of the reactor feed. This may mean that the condensed raw acetic acid (AA) mixture from reactor effluent is processed in the AA unit to separate water from the raw AA mixture. A portion of the separated water along with the condensed water from the process gas compressor area are utilized for the further scrubbing of the AA in a scrubber tower (acetic acid scrubber). The water at the bottom of the tower is recycled back to dilute the mixed feed to the ODH reactor. In contrast, the produced water from AA unit can be directly utilized as the source of water for dilution. However, accommodation of the water from the bottom of AA scrubber should generally be found or specified. If the water from the bottom of AA scrubber were sent to AA unit, that would add significant load to the AA unit. If the water from the bottom of AA scrubber were sent to sewer, that would add load to the wastewater treatment system. Therefore, a closed circuit water system including the water from the bottom of AA scrubber may be a beneficial technique to dilute the feed to the reactor. Lastly, for the water mass balance with respect to the water product discharged from the AA unit, the water product may account for at least the water formed in the ODH reaction in the ODH reactor.

Fourth, utilizing an ethane saturator (e.g., tower or heat exchanger) may allow utilizing lower heat quality source (e.g., LP steam) instead of a higher heat quality source (e.g., MP steam) as typically utilized in DS drum options. This way, more power can be obtained from generated high pressure (HP) or very high pressure (VHP) steam of ODH reactor and boilers when the HP or VHP steam is put into turbines to run the compressors of the plant. As an example (numerical values are only examples and may be different or vary), 1000 kilogram per hour (kg/hr) of VHP going through turbine and extracted as LP steam can generate 145 kilowatts (kW) of power. In contrast, 1000 kg/hr of VHP going through turbine and extracted as MP steam can generate 105 kW of power. This means that to get the similar power of 145 kW extract MP steam is extracted, approximately 40% more VHP steam is consumed than when extracting LP steam in this hypothetical example. The source of LP steam and MP steam can include, for example, extraction turbines or depressurizing valve for HP or VHP steam.

Fifth, a significant heat demand for the ODH reactor system (which can include feed processing and effluent processing) is in the feed dilution area. Evaporating the substantial amount of water to be added to mixed feed utilizes significant amount of heat. In addition, cooling and condensing the added water from reactor effluent utilizes substantial cooling capacity. In embodiments, the heat integration between the feed dilution system and the reactor effluent cooling may beneficially reduce load on steam and cooling tower systems in the ODH reactor plant.

Sixth, the employment of heat exchangers (e.g., as in Options 6 and 7) to saturate the feed without employing a saturator tower may be a beneficial energy approach. As with the saturator tower, a saturator heat exchanger may utilize a low value heat source (e.g., LP steam). However, feed saturation with a saturator heat exchanger may be implemented without pumping the relatively large flow (e.g., 10,000 mg/hr) of water (circulation water) for a saturator tower that gives high power consumption.

Lastly, implementing a dilution steam drum may lead to the most straightforward or simpler technique or approach of Options 1-9 to provide the dilution. However, employing the dilution steam drum (as in Options 8 and 9) utilizes a higher value heat source, such as MP steam, than other options for providing dilution.

Options 1-9 can generally be compared for energy integration of ODH reactor feed saturation with heat recovery via ODH reactor effluent cooling, and with consideration of acetic acid recovery. Table 1 presents simulation results for Options 1-9 in view of the energy integration. In Table 1, Option 1 is utilized as a base case for the comparison. In other words, Options 2-9 may be compared to Option 1 as a baseline case.

Process simulations were developed and performed with Aspen Plus® V10. The SR-POLAR equation of state was utilized for the simulations. For the simulations, the feed inlet temperature (mixed feed 108) into the ODH reactor 102 is maintained below 310° C. at 465 kilopascal (kPa) and oxygen concentration in the mixed feed 108 into the reactor 102 is targeted at 10 volume percent (vol %) in order to be outside the flammability zone. The oxygen to ethane molar ratio in the mixed feed 108 stream is 0.62. Total water content to reactor 102 is 74 vol % which requires heating to evaporate the water before the reactor 102 and cooling to condense the water after the reactor 102. Table 1 shows the impact on heating, cooling, and power of Options 2-9 relative to Option 1 based on different energy integration aspects and strategies. Other aspects of the present techniques fall outside of these example results.

TABLE 1
Comparison of Options 1-9

| | | | | | O2 Sat. Tower | Water Circ. | Reactor |
|---|---|---|---|---|---|---|---|
| | | LPS Amount | MPS Amount | | Water Circ. | Pump(s) Power | Effluent |
| | | for Feed | for Feed | C2 Sat. Tower | Relative to | Consump. for | Cooling |
| | Steam for | Saturation | Saturation | Water Circ. | C2 Sat. Tower | O2/C2 Saturators | Water Duty |
| | Feed | Relative to | Relative to | Relative to | Water Circ. of | Relative to | Relative to |
| Option | Saturation | Option 1 (%) | Option 1 (%) | Option 1 (%) | Option 1 (%) | Option 1 (%) | Option 1 (%) |
| 1 | LP/70 psig | 100% | — | 100% | — | 100% | 100% |
| 2 | LP/60 psig | 101% | — | 55% | 35% | 89% | 106% |
| 3 | LP/60 psig | 69% | — | 100% | 7.5% | 108% | 79% |
| 4 | LP/70 psig | 70% | — | 50% | — | 50% | 77% |
| 5 | LP/70 psig | 63% | — | 50% | — | 50% | 71% |
| 6 | LP/60 psig | 63% | — | — | — | — | 75% |
| 7 | LP/60 psig | 63% | — | — | — | — | 71% |
| 8 | MP/200 psig | — | 66% | — | — | — | 76% |
| 9 | MP/200 psig | — | 80% | — | — | — | 88% |

The comments below based on the process simulations and the results as tabulated in Table 1 are given as examples.

Option 1 utilizes high water circulation and significant amount of LP steam at 70 psig to dilute the mixed feed. Option 1 utilizes a significant amount of cooling water to cool the reactor effluent.

Option 2 has two saturator towers but with less water circulation for each saturator relative to Option 1, which resulted in the same amount of steam but at 60 psig. However, Option 2 has relatively higher cooling-water demand than Option 1.

Option 3 has two saturator towers but with heat recovery mode from reactor effluent. Compared to Option 1, Option 3 utilizes LP steam at 60 psig. However, Option 3 does not have much additional impact on water circulation. The LP steam demand in Option 3 is 31% less than Option 1 and with decrease of cooling water demand of 21% compared to Option 1.

Option 4 has relatively the same energy result as Option 3 but with one saturator tower (for ethane feed) instead of two saturator towers, making Option 3 more straightforward or simpler. However, the LP steam at 70 psig is employed for feed saturation and this results in less water circulation compared to Option 3.

Option 5 includes an additional heat exchanger compared to Option 4. The additional heat exchanger preheats the recycle water for the ethane saturator tower compared to Option 4. The LP steam demand is 37% less than Option 1 with decrease of cooling water demand of 29% compared to Option 1.

Option 6 employs two heat exchangers to fully saturate ethane and oxygen feed against LP steam at 60 psig. This generally removes the need for saturator towers(s) and associate tower water circulation pump(s). The LP steam requirement is 37% less than Option 1 with decrease of cooling water demand of 25% compared to Option 1.

Option 7 employs one heat exchanger to fully saturate the ethane feed. Thus, the heat exchanger may be larger than the heat exchanger utilized for partial saturation of ethane and subsequent heat exchanger for complete saturation of ethane as compared to Option 6. However, use of a heat exchanger for fully saturating the ethane feed with water means less equipment overall compared to employing an ethane saturator tower for fulling saturating the ethane. The LP steam requirement is 37% less than Option 1 with decrease of cooling water demand of 29% compared to Option 1.

Option 8 is one of the simplest ways to dilute the mixed feed using a dilution steam generation system. This utilized MP steam at 200 psig. The MP steam demand is 34% less than the LP steam of Option 1 with decrease of cooling demand of 24% compared to Option 1.

Option 9 may be characterized as the simplest technique of Options 1-9 to dilute the mixed feed. Option 9 is similar to Option 8 but with removal of the two partial saturators of ethane and oxygen feed against reactor effluent. The MP steam requirement is 20% less than Option 1 (LP steam) with 12% less cooling water demand compared to Option 1.

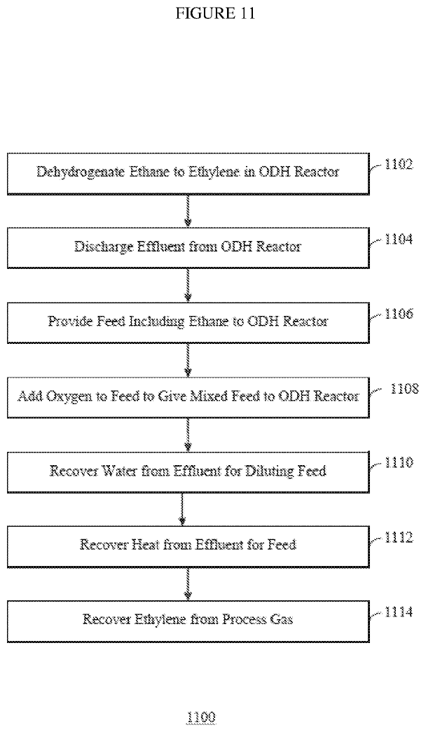

FIG. 11 is a method 1100 of producing ethylene. At block 1102, the method includes dehydrogenating ethane to ethylene via an ODH catalyst in the presence of oxygen in an ODH reactor. The ODH reactor may be, for example, a fixed bed reactor or a fluidized bed reactor. Acetic acid may be produced in the ODH reactor as a byproduct of the ODH reaction that dehydrogenates the ethane to ethylene.

At block 1104, the method includes discharging an effluent from the ODH reactor. The effluent includes ethylene and water, and can include acetic acid, carbon dioxide, carbon monoxide, and unreacted ethane. In some implementations, heat from the effluent can be used to heat water (e.g., boiler feedwater) in a heat exchanger to generate steam for consumption at the facility having the ODH reactor.

At block 1106, the method includes providing feed including ethane to the ODH reactor. The ethane may be ethane gas provided from a supply pipeline or may be recycle ethane from a downstream C2 splitter, and the like. The ethane can be liquid ethane provided from a supply pipeline and that is vaporized to ethane gas. As used herein, the term ethane is generally meant to be ethane gas. As discussed below (block 1110), water (e.g., recycle water) may be added to the ethane gas for feed dilution with the water. In some implementations, the water addition may saturate the ethane gas with the water.

At block 1108, the method includes adding oxygen (O₂ gas) to the feed including the ethane to give a mixed feed to the ODH reactor. As used herein, the term oxygen is generally meant to be O₂ gas. The oxygen may be added at a single addition point to a conduit conveying the feed including the ethane or at multiple addition points to the conduit conveying the feed including the ethane. In some implementations, as discussed below (block 1110), water (e.g., recycle water) may be added to the oxygen for feed dilution with the water prior to addition of the oxygen to the feed. The mixed feed to the reactor includes the ethane gas and the oxygen gas. The mixed feed may include water added for the feed dilution (block 1110).

At block 1110, the method includes recovering water from the effluent as recycle water for diluting the feed to the ODH reactor with the recycle water. As discussed (block 1106), the feed includes ethane. Diluting the feed includes adding the recycle water to the ethane. Diluting the feed may include adding the recycle water to the oxygen added (block 1108) to the feed. The recovering of the water from the effluent for addition to the feed can reduce water consumption in producing the ethylene. In other words, without recovering water from the effluent for feed dilution, external water may need to be consumed for the feed dilution. The recovering of the water from the effluent for the feed dilution can provide for substantial closed-circuit recirculation of the water between the effluent and the feed in some implementations.

The recovering of the water from the effluent can include condensing water and acetic acid in the effluent, such as by routing the effluent through a heat exchanger with cooling water as the cooling medium. The condensed water and condensed acetic acid may be separated from the remaining effluent (gas), such as in a flash drum. The condensed water and condensed acetic in combination can be labeled as raw acetic acid. The remaining effluent (gas) can include ethylene, carbon dioxide, carbon monoxide, and unreacted ethane. The gas can be scrubbed in an acetic acid scrubber (or similar tower) to remove the small amounts of acetic acid vapor and water vapor in the gas. The scrubbed gas may be forwarded as process gas for further processing (block 1114) to recover ethylene as ethylene product. The raw acetic acid can be processed to give acetic acid product, as well as scrubbing water for the acetic acid scrubber. The bottoms stream from the acetic acid scrubber (or similar tower) may be the recycle water utilized for the feed dilution.

The adding of the recycle water to the feed for feed dilution can include adding the recycle water to the ethane in a conduit or in an ethane saturator tower, or both, upstream of adding the oxygen to the feed, or adding the recycle water to the oxygen in a conduit or in an oxygen saturator tower, or both, prior to adding the oxygen to the feed, or any combinations thereof. The adding of the recycle water to the feed for feed dilution can involve vaporizing the recycle water in a steam dilution drum to give dilution steam for addition to the feed. The adding of the recycle water to the feed for feed dilution can include adding the recycle water as dilution steam to the ethane upstream of adding the oxygen to the feed, or adding the recycle water as dilution steam to the oxygen upstream of adding the oxygen to the feed, or a combination thereof.

At block 1112, the method includes recovering heat from the effluent for processing the feed. See above regarding blocks 1106, 1108 for example discussion of the feed. The processing of the feed may include heating the feed. The feed may be heated with the effluent as a heating medium, such as in a heat exchanger (cross-exchanger). Thus, heat may be recovered from the effluent and transferred to the feed in the heat exchanger.

The processing of the feed may include the water dilution (e.g., block 1110) of the feed. The recovering of heat from the effluent for performing the water dilution can include heating the recycle water with heat from the effluent, such as in a cross-exchanger. In implementations, recycle water (e.g., not heated by the effluent) is added to the ethane to give a mixture that is heated (e.g., in a heat exchanger) with heat from the effluent for the water dilution. In some implementations, recycle water (e.g., not heated by the effluent) is added to the oxygen to give a mixture that is heated (e.g., in a heat exchanger) with the effluent. In certain implementations, recycle water is added to the feed comprising ethane comprises adding the recycle water to the oxygen to be added to the feed, and wherein recovering heat from the effluent for performing the water dilution comprises heating a mixture of the recycle water and the oxygen to be added to the feed with heat from the effluent.

The recovering the heat for processing the feed may reduce energy consumption in producing the ethylene. For instance, heating the feed with the effluent may avoid heating the feed with steam, which reduces energy consumption by an amount associated with utilizing the steam. In addition, heating the recycle water (or mixtures having the recycle water) with the effluent may avoid heating the recycle water or mixtures with steam, and therefore avoid the energy consumption associated with using steam for heating the recycle water.

At block 1114, the method includes processing the process gas mentioned with respect to block 1110. The processing can include increasing the pressure of the process gas, such as via a process gas compressor. The processing can include remove light components (e.g., CO) from the process gas. The processing can include separating the ethylene from ethane of the process gas, such as in a C2 splitter (distillation column). The processing of the process gas can give ethylene product for distribution or further processing.

An embodiment includes a method of producing ethylene. The method includes adding water to ethane to give a mixture, flowing the mixture through a feed heat exchanger to heat the mixture with effluent from an ODH reactor, and adding oxygen to the mixture to give a mixed feed for the ODH reactor. The method includes dehydrogenating the ethane to ethylene via an ODH catalyst in the presence of the oxygen in the ODH reactor, and discharging the effluent from the ODH reactor, the effluent including ethylene, water, acetic acid, carbon dioxide, carbon monoxide, and unreacted ethane. The method may include introducing the mixed feed to the ODH reactor, wherein the water added to the ethane includes recycle water from processing of the effluent, and wherein the mixture upstream of the feed heat exchanger includes ethane saturated with the water. The adding of the water to ethane may involve adding water to ethane in an ethane saturator tower. If so, the method may include heating in a cross-exchanger with the effluent the water added to the ethane before adding the water to the ethane in the ethane saturator tower. The adding water to ethane may further include adding water to ethane in a conduit upstream of a heat exchanger upstream of the ethane saturator tower, wherein the heat exchanger is a cross-exchanger that utilizes the effluent as a heating medium.

The adding of the water to ethane may include adding water to ethane in a conduit upstream of a heat exchanger, wherein the heat exchanger is a cross-exchanger that utilizes the effluent as a heating medium. If so, the water may be heated in a second cross-exchanger with the effluent before adding the water to the ethane in the conduit.

The adding of the water to ethane may involve adding dilution steam from a dilution steam drum to a conduit conveying the ethane. If so, the method may include heating the water in a cross-exchanger with the effluent before introducing the water to the dilution steam drum. The adding of water to ethane may further include adding water to a conduit conveying the ethane upstream of a heat exchanger upstream of the ethane receiving the dilution steam, wherein the heat exchanger is a cross-exchanger that utilizes the effluent as a heating medium.

The method may include adding water to the oxygen before adding the oxygen to the mixture, wherein adding the oxygen to the mixture may include adding the oxygen to a conduit conveying the mixture, wherein adding water to the oxygen may involve adding water to a conduit conveying the oxygen upstream of a heat exchanger or adding the water to oxygen in an oxygen saturator tower, or a combination thereof, and wherein the heat exchanger is a cross-exchanger that utilizes the effluent as a heating medium. The adding water to the oxygen may involve adding dilution steam from a dilution steam drum to a conduit conveying the oxygen.

Another embodiment is a method of producing ethylene, including dehydrogenating ethane to ethylene via an ODH catalyst in the presence of oxygen in an ODH reactor, wherein acetic acid is formed in the ODH reactor, and discharging an effluent including ethylene, acetic acid, and water from the ODH reactor. The method includes separating the effluent in a flash drum into gas and raw acetic acid, wherein the gas includes ethylene, water, acetic acid, ethane, carbon dioxide, and carbon monoxide, and wherein the raw acetic acid includes acetic acid and water. The method includes removing acetic acid and water from the gas in an acetic acid scrubber vessel and utilizing a bottoms stream discharged from the acetic acid scrubber vessel as recycle water for diluting feed to the ODH reactor. The method may include heating the recycle water in a cross exchanger with the effluent. The bottoms stream from the acetic acid scrubber as recycle water may include acetic acid in addition to water. The utilizing of the bottoms stream as recycle water for diluting feed to the ODH reactor may include adding the recycle water to ethane. If so, the adding the recycle water to ethane may include adding the recycle water to ethane in an ethane saturator tower or adding the recycle water to a conduit conveying the ethane upstream of a cross exchanger, or a combination thereof, and wherein the cross exchanger utilizes the effluent as a heating medium. The utilizing of the bottoms stream as recycle water for diluting feed to the ODH reactor may include adding the recycle water to oxygen in an oxygen saturator tower or in a conduit upstream of a cross exchanger, or a combination thereof, and wherein the cross exchanger utilizes the effluent as a heating medium.

Yet another embodiment is an ethylene production system including an ODH reactor having an ODH catalyst to dehydrogenate ethane to ethylene and discharge an effluent including ethylene, water, acetic acid, carbon dioxide, carbon monoxide, and ethane. The ethylene production system includes a flash drum to separate effluent from the ODH reactor into gas and raw acetic acid, wherein the gas includes ethylene, water, acetic acid, carbon dioxide, carbon monoxide, and ethane, and wherein the raw acetic acid includes acetic acid and water. The ethylene production system includes an acetic acid scrubber vessel to remove acetic acid and water from the gas and discharge a bottoms stream as recycle water for diluting feed to the ODH reactor, wherein the bottoms stream includes water and acetic acid. The ethylene production system may include a cross exchanger to heat the recycle water with the effluent, and/or a cross-exchanger to receive a mixture of the recycle water and ethane to heat the mixture with the effluent. The ethylene production system may include a cross-exchanger to receive a mixture of the recycle water and oxygen to heat the mixture with the effluent for feed to the ODH reactor.

The ethylene production system may include an ethane saturator tower to receive the recycle water and add the recycle water to ethane for feed to the ODH reactor. If so, the ethylene production system may include a cross exchanger upstream of the ethane saturator tower to heat the recycle water with effluent before addition of the recycle water to the ethane in the ethane saturator tower. In implementations, the ethylene production system may include a cross exchanger upstream of the ethane saturator tower to heat a mixture of the recycle water and ethane with effluent, and a conduit to convey the mixture as heated by the cross exchanger to the ethane saturator tower, wherein the ethane saturator tower receiving the recycle water involves the ethane saturator tower receiving the mixture, and wherein the ethane saturator tower adding the recycle water to ethane involve the ethane saturator tower adding the mixture to ethane in the ethane saturator tower received separately from the mixture. The ethylene production system may include an oxygen saturator tower to receive the recycle water and add the recycle water to oxygen for feed to the ODH reactor.

The ethylene production system may include a steam dilution drum to receive and vaporize the recycle water to dilution steam for addition of the dilution steam to ethane or for addition of the dilution steam to oxygen, or a combination thereof, for feed to the ODH reactor. If so, the ethylene production system may include a cross exchanger upstream of the steam dilution drum to heat the recycle water with effluent before introduction of the recycle water to the steam dilution drum.

Yet another embodiment is a method of producing ethylene, including dehydrogenating ethane to ethylene via an ODH catalyst in the presence of oxygen in an ODH reactor, discharging an effluent (including ethylene, water, acetic acid, carbon dioxide, carbon monoxide, and unreacted ethane) from the ODH reactor, recovering heat from the effluent for processing feed including ethane for the ODH reactor, recovering water from the effluent as recycle water for addition to the feed in performing water dilution of the feed, and adding oxygen to the feed to give a mixed feed including ethane and oxygen to the ODH reactor, wherein the mixed feed includes the water recovered from the effluent as the recycle water and added to the feed. The recovering of the heat from the effluent for processing the feed may reduce energy consumption in producing the ethylene. The recovering of the water from the effluent for addition to the feed may reduce water consumption in producing the ethylene. The recovering of the heat from the effluent for processing the feed may include heating the feed with heat from the effluent. The recovering of the heat from the effluent for processing the feed may include heating the feed in a heat exchanger with the effluent as a heating medium.

The recovering of water from the effluent may involve condensing water and acetic acid in the effluent to give condensed water and condensed acetic acid, and separating raw acetic acid as the condensed water and the condensed acetic acid from the effluent to give a gas including ethylene, carbon dioxide, carbon monoxide, and unreacted ethane from the effluent. Further, the recovering of the water from the effluent may include processing the raw acetic acid to give acetic acid product and scrubbing water, wherein the scrubbing water is for an acetic acid scrubber that removes acetic acid from the gas, and wherein the recycle water includes or is a bottoms stream discharged from the acetic acid scrubber.

The adding of the recycle water to the feed including ethane may involve adding the recycle water to the ethane prior to adding the oxygen to the feed, or involve adding the recycle water to the oxygen prior to adding the oxygen to the feed, or a combination thereof. The adding of the recycle water to the feed including ethane may involve adding the recycle water to the ethane in an ethane saturator tower upstream of adding the oxygen to the feed, or involve adding the recycle water to the oxygen in an oxygen saturator tower prior to adding the oxygen to the feed, or a combination thereof.

The adding of the recycle water to the feed including ethane may involve adding the recycle water as dilution steam to the ethane upstream of adding the oxygen to the feed, or involve adding the recycle water as dilution steam to the oxygen upstream of adding the oxygen to the feed, or a combination thereof. The adding of the recycle water to the feed including ethane may involve vaporizing the recycle water in a steam dilution drum to give dilution steam for addition to the feed.

The processing of the feed may include performing the water dilution of the feed. If so, the recovering of heat from the effluent for performing the water dilution may include heating the recycle water with heat from the effluent. The adding of the recycle water to the feed including ethane may involve adding the recycle water to the ethane to give a mixture, wherein recovering heat from the effluent for performing the water dilution includes heating the mixture with heat from the effluent. The adding of the recycle water to the feed including ethane may involve adding the recycle water to the oxygen to be added to the feed, wherein recovering heat from the effluent for performing the water dilution includes heating a mixture of the recycle water and the oxygen to be added to the feed with heat from the effluent.

Yet another embodiment is a method of producing ethylene, including discharging an effluent (including ethylene, water, acetic acid, carbon dioxide, carbon monoxide, and unreacted ethane) from an ODH reactor that dehydrogenates ethane to ethylene. The method includes performing water dilution of feed including ethane for the ODH reactor. The water dilution includes adding recycle water to the ethane. The method includes recovering water from the effluent to give recovered water as the recycle water for performing the water dilution. The method includes, flowing the feed downstream of the water dilution through a feed heat exchanger to heat the feed with the effluent, and adding oxygen to the feed to give the feed as a mixed feed for the ODH reactor. The performing of the water dilution may further include adding the recycle water to the oxygen. The method may include recovering heat from the effluent for performing the water dilution. The recovering of heat from the effluent for performing the water dilution may include heating the recycle water in a heat exchanger with the effluent as a heating medium. The recovering of heat from the effluent for performing the water dilution may include heating a mixture of the ethane and the recycle water in a heat exchanger with the effluent as a heating medium, or include heating a mixture of the oxygen and the recycle water in a heat exchanger with the effluent as a heating medium, or include both.

A number of implementations have been described. Nevertheless, it will be understood that various modifications may be made without departing from the spirit and scope of the disclosure.

### INDUSTRIAL APPLICABILITY

The present disclosure relates to methods and systems for production of ethylene by oxidative dehydrogenation.

1. A method of producing ethylene, comprising:
adding water to ethane to give a mixture;
flowing the mixture through a feed heat exchanger to heat the mixture with effluent from an oxidative dehydrogenation (ODH) reactor;
adding oxygen to the mixture to give a mixed feed for the ODH reactor;
dehydrogenating the ethane to ethylene via an ODH catalyst in the presence of the oxygen in the ODH reactor; and
discharging the effluent from the ODH reactor, the effluent comprising ethylene, water, acetic acid, carbon dioxide, carbon monoxide, and unreacted ethane.

2. The method of claim 1, comprising introducing the mixed feed to the ODH reactor, wherein the water added to the ethane comprises recycle water from processing of the effluent, and wherein the mixture upstream of the feed heat exchanger comprises ethane saturated with the water.

3. The method of claim 1, wherein adding water to ethane comprises adding water to ethane in an ethane saturator tower.

4. The method of claim 3, comprising heating in a cross-exchanger with the effluent the water added to the ethane before adding the water to the ethane in the ethane saturator tower.

5. The method of claim 3, wherein adding water to ethane further comprises adding water to ethane in a conduit upstream of a heat exchanger upstream of the ethane saturator tower, and wherein the heat exchanger comprises a cross-exchanger that utilizes the effluent as a heating medium.

6. The method of claim 1, wherein adding water to ethane comprises adding water to ethane in a conduit upstream of a heat exchanger, wherein the heat exchanger comprises a cross-exchanger that utilizes the effluent as a heating medium.

7. The method of claim 6, comprising heating the water in a second cross-exchanger with the effluent before adding the water to the ethane in the conduit.

8. The method of claim 1, wherein adding water to ethane comprises adding dilution steam from a dilution steam drum to a conduit conveying the ethane.

9. The method of claim 8, comprising heating the water in a cross-exchanger with the effluent before introducing the water to the dilution steam drum.

10. The method of claim 8, wherein adding water to ethane further comprises adding water to a conduit conveying the ethane upstream of a heat exchanger upstream of the ethane receiving the dilution steam, and wherein the heat exchanger comprises a cross-exchanger that utilizes the effluent as a heating medium.

11. The method of claim 1, comprising adding water to the oxygen before adding the oxygen to the mixture, wherein adding the oxygen to the mixture comprises adding the oxygen to a conduit conveying the mixture.

12. The method of claim 11, wherein adding water to the oxygen comprises adding water to a conduit conveying the oxygen upstream of a heat exchanger or adding the water to oxygen in an oxygen saturator tower, or a combination thereof, and wherein the heat exchanger comprises a cross-exchanger that utilizes the effluent as a heating medium.

13. The method of claim 11, wherein adding water to the oxygen comprises adding dilution steam from a dilution steam drum to a conduit conveying the oxygen.

14. (canceled)

15. The method of claim 1, further comprising:
separating the effluent in a flash drum into gas and raw acetic acid, wherein the gas comprises ethylene, water, acetic acid, ethane, carbon dioxide, and carbon monoxide, and wherein the raw acetic acid comprises acetic acid and water;
removing acetic acid and water from the gas in an acetic acid scrubber vessel;
utilizing a bottoms stream discharged from the acetic acid scrubber vessel as recycle water for diluting feed to the ODH reactor; and
heating the recycle water in a cross exchanger with the effluent.

16-18. (canceled)

19. An ethylene production system comprising:
an oxidative dehydrogenation (ODH) reactor comprising an ODH catalyst to dehydrogenate ethane to ethylene and discharge an effluent comprising ethylene, water, acetic acid, carbon dioxide, carbon monoxide, and ethane;
a flash drum to separate effluent from the ODH reactor into gas and raw acetic acid, wherein the gas comprises ethylene, water, acetic acid, carbon dioxide, carbon monoxide, and ethane, and wherein the raw acetic acid comprises acetic acid and water; and
an acetic acid scrubber vessel to remove acetic acid and water from the gas and discharge a bottoms stream as recycle water for diluting feed to the ODH reactor, wherein the bottoms stream comprises water and acetic acid.

20. The system of claim 19, comprising a cross exchanger to heat the recycle water with the effluent.

21. The system of claim 19, comprising an ethane saturator tower to receive the recycle water and add the recycle water to ethane for feed to the ODH reactor.

22. The system of claim 21, comprising a cross exchanger upstream of the ethane saturator tower to heat the recycle water with effluent before addition of the recycle water to the ethane in the ethane saturator tower.

23. The system of claim 21, comprising:
a cross exchanger upstream of the ethane saturator tower to heat a mixture of the recycle water and ethane with effluent; and
a conduit to convey the mixture as heated by the cross exchanger to the ethane saturator tower, wherein the ethane saturator tower receiving the recycle water comprises the ethane saturator tower receiving the mixture, and wherein the ethane saturator tower adding the recycle water to ethane comprises the ethane saturator tower adding the mixture to ethane in the ethane saturator tower received separately from the mixture.

24. The system of claim 19, comprising a cross-exchanger to receive a mixture of the recycle water and ethane to heat the mixture with the effluent.

25. The system of claim 19, comprising an oxygen saturator tower to receive the recycle water and add the recycle water to oxygen for feed to the ODH reactor.

26. The system of claim 19, comprising a cross-exchanger to receive a mixture of the recycle water and oxygen to heat the mixture with the effluent for feed to the ODH reactor.

27. The system of claim 19, comprising a steam dilution drum to receive and vaporize the recycle water to dilution steam for addition of the dilution steam to ethane or for addition of the dilution steam to oxygen, or a combination thereof, for feed to the ODH reactor.

28. The system of claim 27, comprising a cross exchanger upstream of the steam dilution drum to heat the recycle water with effluent before introduction of the recycle water to the steam dilution drum.

29. (canceled)

30. The method of claim 1, further comprising:
recovering heat from the effluent for processing mixed feed; and
recovering water from the effluent as recycle water for addition to the mixed feed, wherein recovering the heat reduces energy consumption in producing the ethylene, and wherein recovering the water reduces water consumption in producing the ethylene.

31. The method of claim 30, wherein recovering heat from the effluent for processing the feed comprises heating the feed with heat from the effluent.

32. The method of claim 30, wherein recovering heat from the effluent for processing the feed comprises heating the feed in a heat exchanger with the effluent as a heating medium.

33. The method of claim 30, wherein recovering water from the effluent comprises condensing water and acetic acid in the effluent to give condensed water and condensed acetic acid, and separating raw acetic acid comprising the condensed water and the condensed acetic acid from the effluent to give a gas comprising ethylene, carbon dioxide, carbon monoxide, and unreacted ethane from the effluent.

34. The method of claim 33, wherein recovering water from the effluent comprises processing the raw acetic acid to give acetic acid product and scrubbing water, wherein the scrubbing water is for an acetic acid scrubber that removes acetic acid from the gas, and wherein the recycle water comprises a bottoms stream discharged from the acetic acid scrubber.

35. The method of claim 30, wherein adding the recycle water to the feed comprising ethane comprises adding the recycle water to the ethane prior to adding the oxygen to the feed, or wherein adding the recycle water to the feed comprising ethane comprises adding the recycle water to the oxygen prior to adding the oxygen to the feed, or a combination thereof.

36. The method of claim 30, wherein adding the recycle water to the feed comprising ethane comprises adding the recycle water to the ethane in an ethane saturator tower upstream of adding the oxygen to the feed, or wherein adding the recycle water to the feed comprising ethane comprises adding the recycle water to the oxygen in an oxygen saturator tower prior to adding the oxygen to the feed, or a combination thereof.

37. The method of claim 30, wherein adding the recycle water to the feed comprising ethane comprises adding the recycle water as dilution steam to the ethane upstream of adding the oxygen to the feed, or wherein adding the recycle water to the feed comprising ethane comprises adding the recycle water as dilution steam to the oxygen upstream of adding the oxygen to the feed, or a combination thereof.

38. The method of claim 30, wherein adding the recycle water to the feed comprising ethane comprises vaporizing the recycle water in a steam dilution drum to give dilution steam for addition to the feed.

39. The method of claim 30, wherein processing the feed comprises performing the water dilution of the feed.

40. The method of claim 39, wherein recovering heat from the effluent for performing the water dilution comprises heating the recycle water with heat from the effluent.

41. The method of claim 39, wherein adding the recycle water to the feed comprising ethane comprises adding the recycle water to the ethane to give a mixture, and wherein recovering heat from the effluent for performing the water dilution comprises heating the mixture with heat from the effluent.

42. The method of claim 39, wherein adding the recycle water to the feed comprising ethane comprises adding the recycle water to the oxygen to be added to the feed, and wherein recovering heat from the effluent for performing the water dilution comprises heating a mixture of the recycle water and the oxygen to be added to the feed with heat from the effluent.

43. A method of producing ethylene, comprising:
discharging an effluent from an oxidative dehydrogenation (ODH) reactor that dehydrogenates ethane to ethylene, the effluent comprising ethylene, water, acetic acid, carbon dioxide, carbon monoxide, and unreacted ethane;
performing water dilution of feed comprising ethane for the ODH reactor, wherein the water dilution comprises adding recycle water to the ethane;
recovering water from the effluent to give recovered water as the recycle water for performing the water dilution;
flowing the feed downstream of the water dilution through a feed heat exchanger to heat the feed with the effluent; and
adding oxygen to the feed to give the feed as a mixed feed for the ODH reactor.

44. The method of claim 43, wherein performing the water dilution further comprises adding the recycle water to the oxygen.

45. The method of claim 43, comprising recovering heat from the effluent for performing the water dilution.

46. The method of claim 45, wherein recovering heat from the effluent for performing the water dilution comprises heating the recycle water in a heat exchanger with the effluent as a heating medium.

47. The method of claim 45, wherein recovering heat from the effluent for performing the water dilution comprises heating a mixture of the ethane and the recycle water in a heat exchanger with the effluent as a heating medium, or heating a mixture of the oxygen and the recycle water in a heat exchanger with the effluent as a heating medium, or both.
